(19) United States
(12) Patent Application Publication
(10) Pub. No.: US 2021/0196960 A1
(43) Pub. Date: Jul. 1, 2021

(54) PHYSIOLOGICAL MEASUREMENT MANAGEMENT UTILIZING PROSTHESIS TECHNOLOGY AND/OR OTHER TECHNOLOGY

(71) Applicant: Cochlear Limited, Macquarie University, NSW (AU)

(72) Inventors: Kenneth OPLINGER, Macquarie University (AU); Stefan Jozef MAUGER, Macquarie University (AU)

(21) Appl. No.: 17/269,618

(22) PCT Filed: Nov. 1, 2019
(86) PCT No.: PCT/IB2019/059403

§ 371 Date: Feb. 19, 2021

Related U.S. Application Data
(60) Provisional application No. 62/754,776, filed on Nov. 2, 2018.

(51) Int. Cl. A61N 1/36; A61N 1/02; G16H 40/67

(52) U.S. Cl. CPC A61N 1/36139; A61N 1/36039; A61B 5/369; G16H 40/67; A61N 1/36046; A61N 1/025

(57) ABSTRACT

A medical device, wherein the medical device is configured to determine whether or not a data collection activity should be commenced, wherein the data is physiological data associated with a recipient of the medical device. In an exemplary embodiment, the medical device can be a prosthesis, such as an implanted prosthesis, and in other embodiments, the medical device is different from a prosthesis.

### BACKGROUND

This application claims priority to U.S. Provisional Application No. 62/754,776, entitled PHYSIOLOGICAL MEASUREMENT MANAGEMENT UTILIZING PROSTHESIS TECHNOLOGY AND/OR OTHER TECHNOLOGY, filed on Nov. 2, 2018, naming Kenneth OPLINGER of Macquarie University, Australia as an inventor, the entire contents of that application being incorporated herein by reference in its entirety.

### BACKGROUND

People suffer from sensory loss, such as, for example, eyesight loss, hearing loss, hyposmia, etc. With respect to hearing loss, such may be due to many different causes, generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional acoustic hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve.

Cases of conductive hearing loss can be treated by means of bone conduction devices. In contrast to conventional hearing aids, these devices use a mechanical actuator that is coupled to the skull bone to apply the amplified sound. Other types of devices, such as middle ear implants, can be utilized to evoke a hearing percept to address conductive hearing loss.

In contrast to hearing aids, which rely primarily on the principles of air conduction, certain types of hearing prostheses, commonly referred to as cochlear implants, convert a received sound into electrical stimulation. The electrical stimulation is applied to the cochlea, which results in the perception of the received sound.

Moreover, some people can often be totally blind or otherwise legally blind. Retinal implants can provide stimulation to a recipient to evoke a sight percept. In some instances, the retinal implant is meant to partially restore useful vision to people who have lost their vision due to degenerative eye conditions such as retinitis pigmentosa (RP) or macular degeneration. In some instances, not mutually exclusive with the aforementioned instances at least in some instances, a retinal implant is provided to provide at least a modicum of spatial perception and/or situational awareness to a person who otherwise cannot see.

Typically, there are three types of retinal implants that can be used to restore partial sight: epiretinal implants (on the retina), subretinal implants (behind the retina), and suprachoroidal implants (above the vascular choroid). Retinal implants provide the recipient with low resolution images by electrically stimulating surviving retinal cells. Such images may be sufficient for restoring specific visual abilities, such as light perception and object recognition.

Still further, other types of sensory loss entail somatosensory and chemosensory deficiencies. There can thus be somatosensory implants and chemosensory implants that can address such.

The various prostheses described above sometimes utilize sophisticated processing (e.g., sound processing, image processing, etc.) techniques so as to improve the evoked percept (hearing, vision, etc.) relative to that which would otherwise be the case.

Many devices, such as medical devices that interface with a recipient, have structural and/or functional features where there is utilitarian value in adjusting such features for an individual recipient. One type of medical device where there is utilitarian value in making such adjustments is the above-noted cochlear implant. That said, other types of medical devices, such as other types of hearing prostheses, and other types of prostheses, such as a retinal implant, exist where there is utilitarian value in fitting such to the recipient.

### SUMMARY

In an exemplary embodiment, there is a medical device, wherein the medical device is configured to determine, based on non-movement data associated with a recipient of the medical device, whether or not a data collection activity should be commenced, wherein the data is physiological data associated with the recipient of the medical device.

In an exemplary embodiment, there is a method, comprising obtaining first data indicative of an occurrence associated with a recipient of a prosthesis utilizing a device of the recipient and determining whether to at least one of implement measuring involving the recipient or to discount second data involving the recipient based on the obtained first data.

In an exemplary embodiment, there is a system, comprising a first sub-system configured to sense a phenomenon associated with an individual, a second sub-system configured to at least one of capture sound, capture light, or capture electromagnetic radiation and a third sub-system configured to at least one of:

analyze output from at least the second sub-system and determine at least one of whether to activate the first sub-system or a level of activation of the second sub-system; or

analyze output from at least the second sub-system and the first sub-system and determine at least one of whether to activate or a level of activation of a fourth sub-system that stimulates the recipient.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIG. 2 presents a functional block diagram of an example cochlear implant;

FIG. 3A and FIG. 3B present exemplary systems according to some embodiments;

FIG. 4 presents an exemplary external component;

FIGS. 5 and 6 and 7 present schematics of some exemplary body monitoring systems;

FIG. 8 presents an exemplary sensory prosthesis;

FIGS. 9, 10 and 11 provide exemplary algorithms for exemplary methods;

FIG. 12 presents a functional diagram of an exemplary system; and

FIGS. 13-16 present schematics of some exemplary body monitoring systems.

### DETAILED DESCRIPTION

The teachings detailed herein are implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

While the teachings detailed herein will be described for the most part with respect to a hearing prosthesis, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system, and/or method that is used to or results in ultimate stimulation of the auditory nerve corresponds to a disclosure of an analogous stimulation of the optic nerve utilizing analogous components/methods/systems.

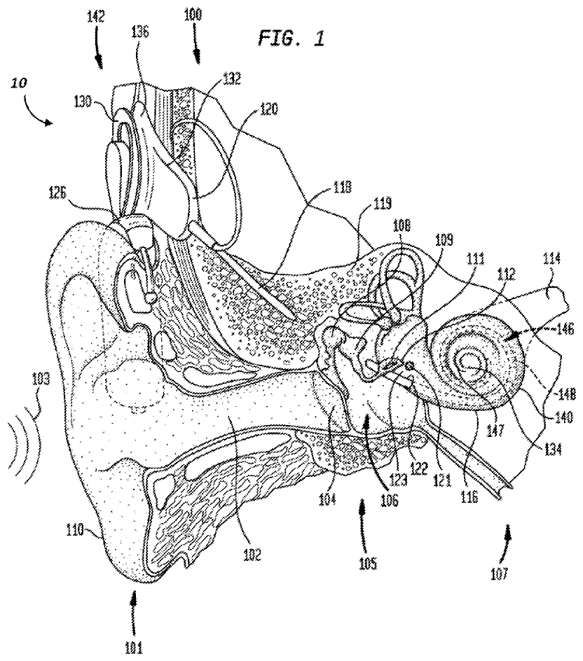

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In this regard, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically stimulating auditory prostheses (e.g., auditory brain stimulators), etc. The techniques presented herein may also be used with visual prostheses (i.e., Bionic eyes), sensors, pacemakers, drug delivery systems, defibrillators, functional electrical stimulation devices, catheters, etc. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. The teachings detailed herein are applicable, in at least some embodiments, to partially implantable and/or totally implantable medical devices that provide a wide range of therapeutic utility to recipients, patients, or other users, e.g., hearing devices having an implanted microphone, auditory brain stimulators, visual prostheses (e.g., bionic eyes), sensors, etc.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

Technologies disclosed herein can be used to provide power to and/or data to and/or retrieve data from an implantable device in situations where a recipient is not wearing an external device. The technologies can overcome one or more challenges associated therewith. In an example, disclosed technologies can provide a source of power and/or data for an implanted medical device via a system that includes a pillow or other headrest or other bodyrest component (mattress, blanket, etc.). Disclosed technologies can be configured to continuously and/or intermittently provide power and data to an implantable medical device over a period of time (e.g., substantially the entire period of time where the recipient is resting their head on the pillow). Characteristics of the continuously provided power need not be constant. For example, the power may fluctuate because the efficiency of the link between the implant and the pillow may vary as the recipient's head moves, causing the proximity of the coils to vary. The power to the implanted electronics can be smoothed for example using tank capacitors. It is common for recipients of an implanted medical device to remove their external devices while sleeping and during that time pillows are often placed in close proximity to the implanted prosthesis. In particular, auditory implants are typically disposed in close proximity to a recipient's ears and people typically place their head on a pillow such that one or both ears are close to the pillow. Thus, it can be utilitarian to incorporate a pillow into a system for providing functionality of a worn external device while a recipient of an implantable device is sleeping. For a recipient of bilateral auditory implants, it may be sufficient for night time use for only one of the two devices to function. For instance, a first device being closest to the pillow may receive sufficient power and/or data to function while a second device that is further away from the pillow may receive insufficient power and/or data to function.

Reference may be made herein to pillows or other headrests for concision, but disclosed technologies can be can be used in conjunction with a variety of articles. Headrests can include, for example, pillows, cushions, pads, head supports, and mattresses, among others. Such articles may be covered (e.g., with a pillow case) or uncovered. Additionally, the disclosed external system components can be used with any of a variety of systems in accordance with embodiments of the technology. For example, in many embodiments, the technology is used in conjunction with a conventional cochlear implant system. FIG. 1 depicts an exemplary cochlear implant system that can benefit from use with technology disclosed herein.

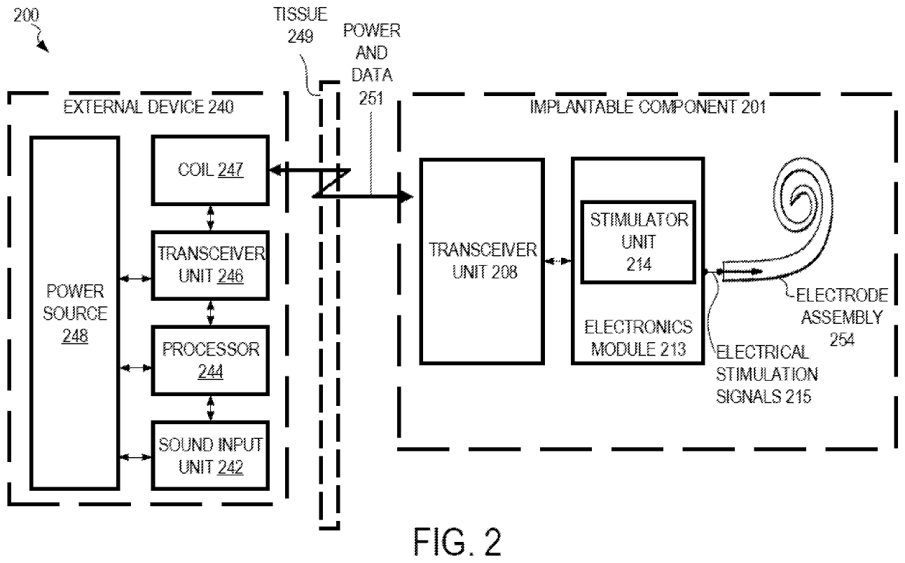

FIG. 2 is a functional block diagram of a cochlear implant system 200 that can benefit from the use of a pillow system in accordance with certain examples of the technology described herein. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

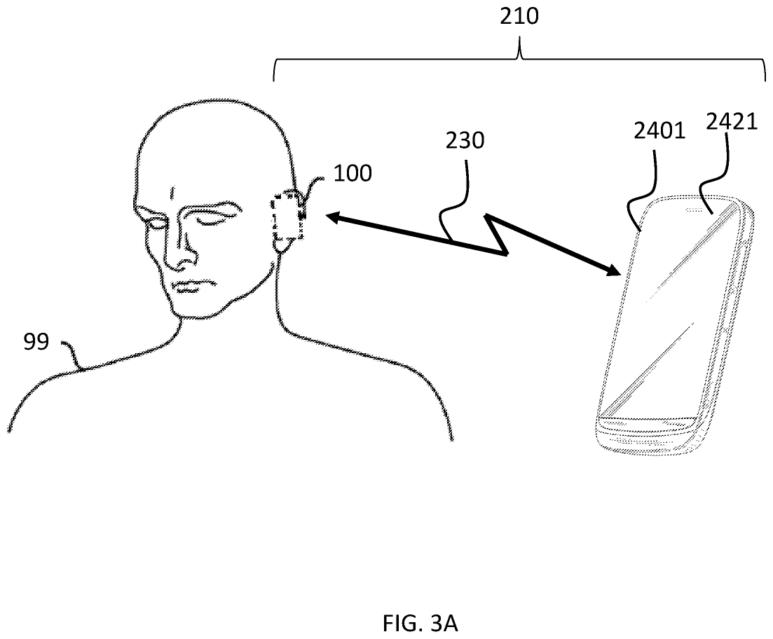

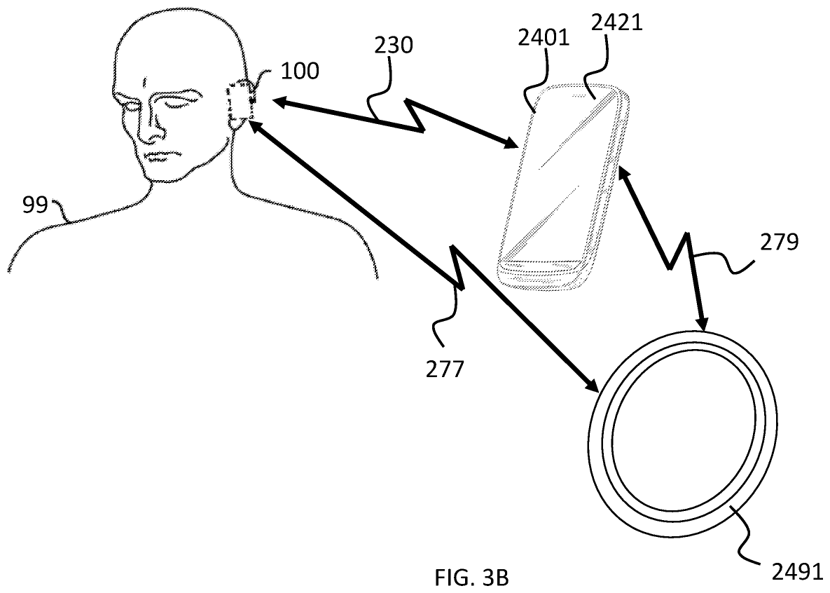

FIG. 3A depicts an exemplary system 210 according to an exemplary embodiment, including hearing prosthesis 100, which, in an exemplary embodiment, corresponds to cochlear implant 100 detailed above, and a portable body carried device (e.g. a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).)

In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Sep. 15, 2018. In an exemplary embodiment, the device 2401 corresponds to a Samsung Galaxy Gear™ Gear 2, as is available in the United States of America for commercial purchase as of Sep. 15, 2018. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 2B depicts an exemplary quasi-functional schematic depicting communication between an external communication system 2491 (e.g., a telecoil), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)).

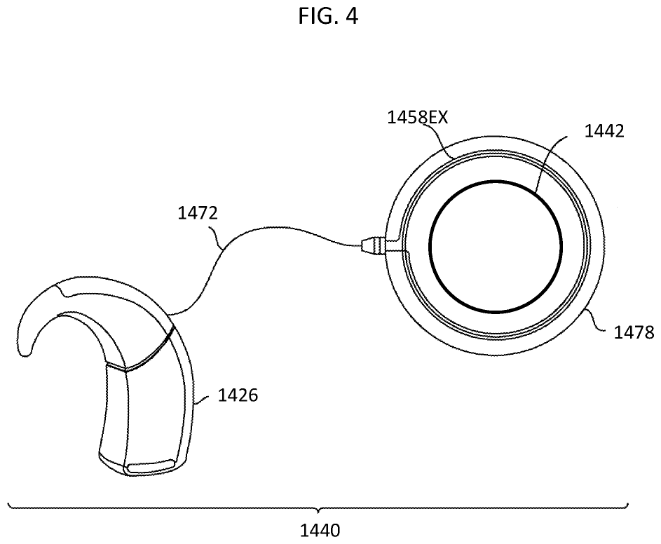

It is noted that while some embodiments detailed herein are described in terms of utilizing an external device that is fixed or otherwise relatively immobile (e.g., a device integrated into a bed, for example) or a device that can be in a relatively easily movable object (a pillow, a shirt, etc.), to communicate and/or power the implanted component, it is to be understood that these devices can also be powered by their traditional external components and/or communicated therewith via their traditional external components. In this regard, FIG. 4 depicts an exemplary external component 1440. External component 1440 can correspond to external component 142 of the system 10. As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

Accordingly, in an exemplary embodiment, external component 1440 can be utilized with the implantable component that is an implantable hearing prosthesis and/or an implantable retinal implant and/or an implantable sense prosthesis as detailed herein where the implanted coil is implanted near or in the head.

In some embodiments, with respect to any of the devices detailed herein and/or variations thereof, there can be utilitarian value with respect to measuring a physiological feature of the user. In the case of cochlear implants, in an exemplary embodiment, the electrically evoked compound action potential in response to stimulating the cochlea can be measured. In another example, the EEG of the patient/recipient is measured. Many physiological and environmental factors can influence recordings. There is benefit in understanding the factors when measuring a physiological feature of a user/recipient.

In an exemplary embodiment, there can be sensors, such as implanted or internal sensors, that can be utilitarian in at least partially aiding in a process that includes determining a temporal period when it might be utilitarian to take a measurement of something associated with a person. By way of example only and not by way of limitation, in an embodiment where there is the execution of acoustic probing (e.g., via the utilization of any of the hearing prostheses disclosed herein that can enable such, including a conventional hearing aid, or the utilization of a non-prosthetic device, such as the speaker of a smart phone or smart device, etc.) to obtain data related to a recipient based on a reaction or a response to a probe or any other utilitarian phenomenon that can be detected, for example, a microphone could be used to obtain data that can be used to ascertain, directly and/or through latent variables, that there exists an environment where external noise is at a level and/or below a level such that the use of the acoustic probe and/or the data resulting from such utilization can be used with a minimum of efficacy (e.g., that the person can hear the sound generated by the acoustic probe vs. the ambient noise). In an exemplary embodiment, acoustic probing is executed using an acoustic-type signal and/or an acoustic simulating signal, while in other embodiments, the acoustic signal is a purely acoustic signal, while in other embodiments, the acoustic-type signal excludes a purely acoustic signal. In some embodiments, the probing is probing using electrical stimulation of tissue, etc.

In view of the above, there is an apparatus, comprising a medical device, such as any of the medical devices disclosed herein, such as, for example, the cochlear implant above and/or a conventional hearing aid, or a retinal implant, etc. In this exemplary embodiment, the medical device is configured to determine whether or not a data collection activity should be commenced, wherein the data is physiological data associated with a recipient of the medical device. In an exemplary embodiment where the medical device is a hearing prosthesis, the hearing prosthesis is configured to evaluate a sound environment of the hearing prosthesis to determine whether or not the data collection activity should be commenced. By way of example only and not by way of limitation, in an exemplary embodiment, there is the utilization of a hearing prosthesis in accordance with at least some of the teachings detailed herein, in conjunction with data collection associated with recording electroencephalogram (EEG) data. Indeed, in an exemplary embodiment, an acoustic transducer, or any other sound creating or a hearing percept evoking device that can be utilized, is utilized to evoke a hearing percept. By way of example only and not by way of limitation, this can be done utilizing an implanted actuator, such as a bone conduction device, or a middle ear implant. Still further, this can be done utilizing a cochlear implant, an auditory brainstem implant, or an auditory midbrain implant, etc. In an alternate embodiment, a conventional hearing aid can be utilized to evoke the hearing percept. In some embodiments, a combination of two or more of the aforementioned devices can be utilized to evoke the hearing percept. Other devices can be utilized as well. In an exemplary embodiment, the above-noted portable handheld device 2401 can be utilized, or a noise or sound producing device specifically designed and fabricated for medical procedures. In an exemplary embodiment, the determination is made based on whether the recipient is moving or not, while in other embodiments, the movement is determined based on non-movement data/data that is not related to movement data (e.g., data that is not based on the output of an accelerometer and/or a device that determines that the recipient is moving, as different from a device that can determine that the recipient has moved/is in a new location than that which was previously the case). To be clear, “non-movement data” as used herein means that the data is unrelated to movement, not that the recipient is not moving.

In many instances, the teachings detailed herein will be directed towards implantable components and the like and/or prostheses. It is noted that any disclosure herein of an implantable component corresponds to an alternative disclosure of an apparatus or a component that is not implanted that has the functionality that is the same as or sufficiently efficaciously similar to the implanted component. Further, any disclosure herein of a prosthesis corresponds to an alternative disclosure of an apparatus or a component that is not a prostatic component. Any disclosure herein of a prosthesis corresponds to an alternative disclosure of a body worn or body carried device. Note further, any disclosure of a body worn or body carried device and/or a prosthesis and/or an implanted component corresponds to a disclosure of a device that is stationary or semi-stationary that has that functionality. All of this is contingent upon the art enabling such, as well as any explicit proviso detailed herein stating that such is not the case.

In many instances, implanted devices, such as implanted electrodes, are disclosed herein for utilization with respect to monitoring physiological characteristics. Consistent with the aforementioned statements in the above paragraph, any disclosure herein of an implanted component that is utilized for measurements or sensation purposes also corresponds to an alternate disclosure of a device and/or an apparatus for component that is not implanted but has that functionality or otherwise enable such functionality, again subject to the aforementioned provisos.

In this exemplary embodiment under discussion, the sound that is produced or otherwise the stimulus that is utilized to evoke a hearing percept are utilized to elicit a time specific response in the brain, such as an EEG response. In this regard, sound, or the perceptive sound, can cause the brain to be stimulated and thus produce brain waves which can be detected/recorded and the data associated therewith can be analyzed, sometimes in real time, to evaluate the state of a person's brain.

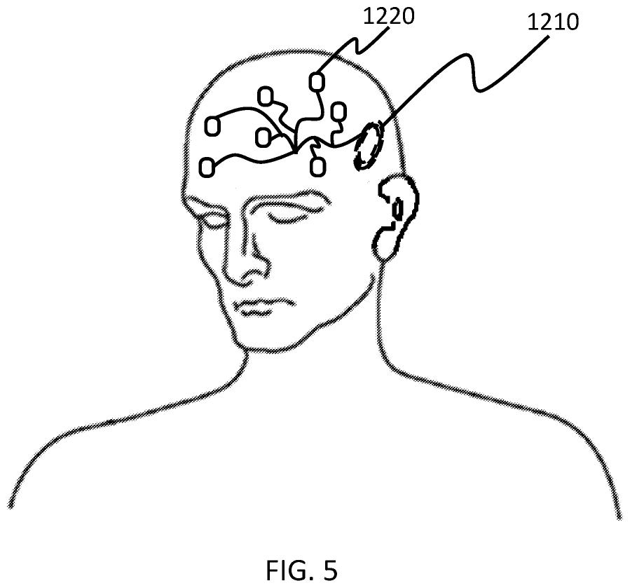

FIG. 5 provides an exemplary embodiment of an EEG system that is implanted in the recipient, where read/sense electrodes 1220 are arrayed inside a recipient's head and in signal communication with a coil 1210 via electrical leads. In this embodiment, the implanted device has no recording/storage capabilities, and requires an external device to receive a signal from the implanted inductance coil 1210 so as to retrieve in real time the signal therefrom. Not shown is an implantable component that converts the electricity sensed by the sensor/read electrodes into a signal that is transmitted by the inductance coil 1210. In an exemplary embodiment, the sensor arrangement seen in FIG. 5 is an implanted EEG sensor arrangement.

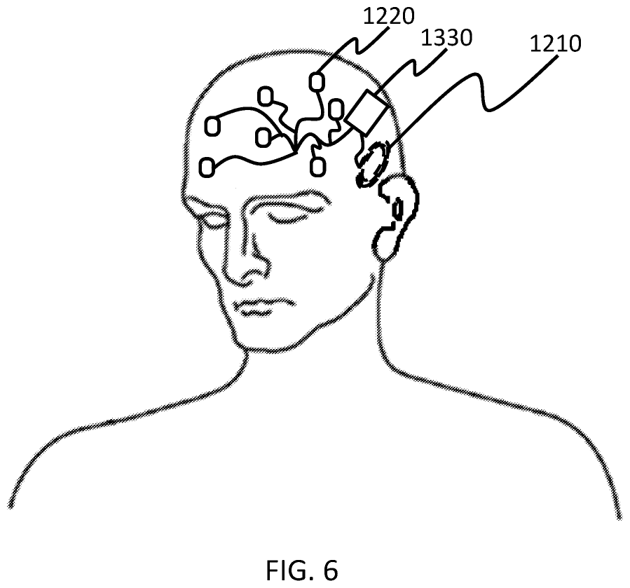

FIG. 6 depicts another arrangement of an implantable sensor arrangement that again includes the sensor/read electrodes 1220 and the leads. Here, in this embodiment, there is a housing 1330 which includes circuitry that is configured to receive the signals from the leads from the electrodes 1220 and record the data therefrom or otherwise store the data, and permits the data to be periodically read from an external device when the external device comes into signal communication with the implanted inductance coil 1210. Alternatively, and/or in addition to this, the circuitry is configured to periodically energize the inductance coil 1210 so as to provide the data to the coil 1210 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EEG sensor arrangements can be obtained and modified so as to implement the teachings detailed herein and/or variations thereof.

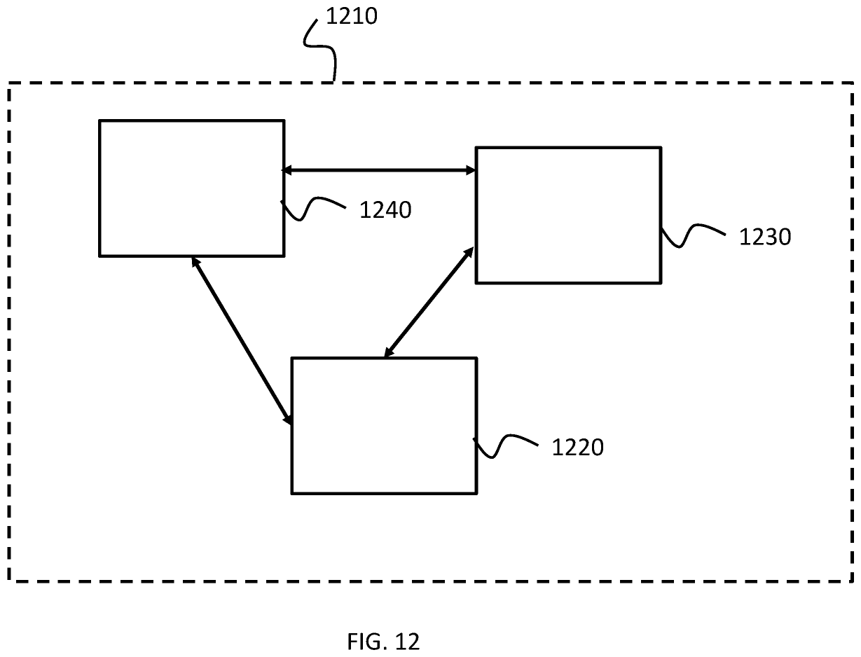

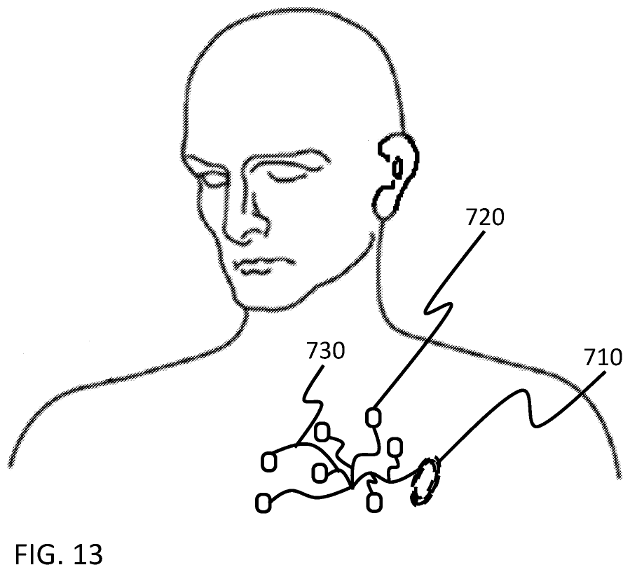

It is noted that some embodiments of the sensor arrangement of FIG. 13 include an implanted battery or otherwise implanted power storage arrangement, while in other embodiments the arrangement specifically does not, making the arrangement akin to the embodiment of FIG. 12.

In view of the above, it is to be understood that in at least some exemplary embodiments, there are traditional implanted EEG and EKG sensor systems that are configured to communicate with the external devices detailed herein (e.g., the device of FIG. 4, or the aforementioned “pillow chargers” or “bed chargers,” etc.). In an exemplary embodiment, the structure implanted in the recipient is the exact same thing as these traditional sensor systems, with the exception that they have been modified to operate in the various modes detailed herein, such as by way of programming or by structural modification or by the inclusion of logic circuitry, etc. That is, in an exemplary embodiment, the sensory systems of FIGS. 5 and 6 are used in combination with the pillow charger detailed above for communication and/or powering and/or charging. Any disclosure herein of the use of the pillow charger associated with the hearing prosthesis detailed above also corresponds to the use of the pillow charger for data transfer and/or for powering and/or charging the sensor systems of FIGS. 5 and 6 or any other sensor systems detailed herein, just as any disclosure associated with the pillow charger vis-à-vis the cochlear implant also corresponds to a disclosure of such with respect to an implanted middle ear prosthesis, a DACI and an active transcutaneous bone conduction device.

Returning back to the feature that began the aforementioned discussion about EEG, the embodiment where the medical device is a hearing prosthesis, and the hearing prosthesis is configured to evaluate a sound environment of the hearing prosthesis to determine whether or not the data collection activity should be commenced, the microphone of the hearing prostheses, whether it is an external microphone or an implanted microphone, or even a microphone that is not part of the hearing prostheses per se, is a microphone that can be utilized to communicate with the hearing prostheses, captures the ambient sound. The prosthesis is configured to evaluate the ambient sound, or more accurately, evaluate/analyze the data (signal data) outputted by the sound capture device (microphone) and deduce a current sound environment. By way of example only and not by way of limitation, a signal to noise ratio can be developed or otherwise identified based on the data from the microphone. An absolute sound level/average sound level, such as 77 dB, 40 dB, 100 dB, etc., can be derived based on the data from the microphone. Utilizing predetermined algorithms or otherwise data, such as that embodied in a lookup table, a comparison between the results of the analysis can be made to the predetermined data to determine the level of quietness or loudness, etc., of an environment, based on the data. In this regard, in an exemplary embodiment, the hearing prosthesis can include a processor or otherwise logic circuits or some other form of circuitry that can put on the aforementioned analysis.

In an exemplary embodiment, the devices/apparatuses herein are configured to identify the existence and/or the absence of at least one of environmental sound, environmental light, electromagnetic radiation or a magnetic field that at least one of meets or does not meet a predetermined criteria and determine whether or not the data collection activity should be commenced based on the identified existence and/or absence and/or are configured to evaluate at least one of an intensity, spectrum or fluctuation of environmental sound and/or light and determine whether such meets and/or does not meet a predetermined criteria and determine whether or not the data collection activity should be commenced based on the identified existence and/or absence.

The hearing prosthesis can determine whether or not the data collection activity should be commenced and/or whether data should be discounted or not based on the analysis, or otherwise provide output/data that can enable such a determination. In an exemplary embodiment, if the ambient sound is fluctuating, such that the signal level is changing more than 5, 6, 7, 8, 9, 10, 11, 12, 13, 14, 15, 16, 17, 18, 19 or 20 dB for example over a given period of time, such as within, more than or less than 0.25, 0.5,0.75, 1, 1.25, 1.5, 1.75, 2, 2.5, 3, 3.5, 4, 4.5 or 5 seconds, for instance, the prosthesis may determine that data collection should not be commenced, because, for example, the ambient sound would evoke a reaction in the brain resulting in EEG signals that would obfuscate the brain's general activity not related to the ambient sound decreasing the utilitarian value of the EEG when analyzed for the purposes of the analysis (e.g., the magnitudes of the signals will change partly in response to the ambient sound, which might be sufficient to confound the utilitarian value or the EEG response with respect the its general activity of which the EEG signals of interest).

The hearing prosthesis can determine whether or not the data collection activity should be commenced based on the analysis. In a further exemplary embodiment, if the ambient sound is determined to be at a level of 100 dB, the prosthesis may determine that data collection should not be commenced, because, in the embodiment where a sound source is utilized to evoke a reaction in the brain, it is deemed that the recipient of the hearing prosthesis may not be able to hear the sound, or otherwise even if the recipient hears the sound or otherwise even distinguishes the sound from the ambient environment, the resulting EEG signals will not be utilitarian when those are analyzed for the purposes of the analysis (e.g., the magnitudes of the signals will change only slightly from that which was the case prior to the commencement of data collection, or more accurately, prior to the commencement of the additional noise or otherwise the action of evoking the hearing percept for the purposes of stimulating the brain).

Various devices, systems, and methods exist that enable an ambient sound environment to be analyzed. These can be included in the hearing prosthesis by way of dedicated specific circuitry added thereto, or by way of modifying the existing circuitry thereof (e.g., reprogramming the existing processors, etc.).

It is noted that with respect to the configuration that is utilized to evaluate a sound environment of the hearing prosthesis to determine whether or not data collection activity should be commenced, in some embodiments, the hearing prosthesis is configured to then commence the data collection activity. In this regard, in an exemplary embodiment, such as for example only and not by way of limitation, where probing in used, external component 1440 can output a sound from any of the speaker devices/receiver devices associated there with, such as in the case of an in the ear canal speaker with respect to a conventional hearing aid, or to cause an implanted actuator to operate to evoke a hearing percept, or to cause an implanted cochlear implant electrode array, etc., to provide stimulation to the cochlea to evoke a hearing percept, etc. Further, in an exemplary embodiment, the external component 1440 can be configured to communicate with the implanted coil 1210, to extract the data from the recipient that was recorded or otherwise collected by the read electrodes 1220, etc. That said, in an exemplary embodiment, the external component 1440 is not configured to so communicate. Instead, a separate device can be utilized to retrieve the data. In this regard, the device that is utilized to retrieve the data can be a completely separate component from the hearing prostheses or otherwise the medical device that was utilized to evaluate the sound environment. Indeed, it is noted that in some exemplary embodiments, it is not even the hearing prostheses that is utilized to evoke the hearing percept or otherwise to generate sound. In an exemplary embodiment, as noted above, a separate device can be utilized. That said, there can be utilitarian value with respect to combining the devices, at least with respect to the device that analyzes a sound environment and the device that causes the hearing percept to be evoked so as to stimulate the brain.

In view of the above, it can be seen that in some embodiments, the hearing prosthesis does not necessarily engage in any of the affirmative actions associated with the data collection activity. In this regard, in an exemplary embodiment, the hearing prosthesis can simply output data indicative of the results of the analysis. By way of example only and not by way of limitation, in an exemplary embodiment, such as where the recipient is an active participant in the data collection activity, the prosthesis can indicate to the recipient something like “now is a good time to perform brain monitoring”. This could be an artificial voice that would be produced by the hearing prosthesis. With respect to the implantable hearing prosthesis, it could be something that only the recipient can hear. In an exemplary embodiment of this exemplary embodiment, the recipient can then affirmatively engage the testing regime. In an exemplary embodiment, the recipient can close their eyes and relax momentarily. Or further in an exemplary embodiment, the recipient can affirmatively engage the test regime when the recipient is in a suitable physical state and expectant of the test when the sound is produced or otherwise the hearing percept is evoked, and the data collection activity is commenced. This could be something as simple as the recipient closing their eyes (and affirmative detection of this activity by the measurement device) or the recipient saying “go for it” or something along those lines within a limited temporal period from the aforementioned notice from the hearing prosthesis. This can also be the recipient affirmatively pressing a button or the like on the external component, where the recipient could activate an application that is on the portable handheld electronics device 241 to implement the testing, etc. Any device, system, and/or method that can enable the teachings detailed herein can be utilized in at least some exemplary embodiments.

Note also that in at least some exemplary embodiments, the hearing prosthesis does not communicate with the recipient, at least not directly. Instead, in an exemplary embodiment, after the evaluation of the sound environment, the hearing prosthesis can communicate with a remote device, such as the portable handheld electronics device 2401 detailed above, which could be in control of the overall effort to provide the hearing prostheses to revoke the brain stimulation. That said, the hearing prosthesis can communicate with a remote device that is located distant with respect to geography, such as a remote server or the like, which could control the data collection activity and the testing, etc.

Still, at least some exemplary embodiments include an integrated system and/or a semi-integrated system of which the hearing prosthesis is a part, which integrated system and/or semi-integrated system can perform at least one or more or all of the method actions detailed herein or otherwise has the functionality of one or more or all of the functionalities detailed herein.

Thus, it can be seen that in at least some exemplary embodiments, the medical device can be configured to execute EEG monitoring. In an exemplary embodiment, the data collection activity is EEG signal collection/recording/reading. As will be detailed below, the data collection activity can instead be EKG signal collection/recording/reading. The data collection activity can also ibe single action potentials, multi-unit cluster recordings, compound action potentials, or other neural responses. Other types of data collection activities can be utilized. Any data collection activity that can have utilitarian value with respect to analyzing a feature associated with the body of the recipient can be utilized in at least some exemplary embodiments.

Concomitant with the embodiments detailed above associated with the EEG/EKG monitoring, in an exemplary embodiment, the data collection activity is data collection utilizing an implanted electrode that is part of the medical device. It is noted that in at least some exemplary embodiments, electrodes of the cochlear electrode array can be utilized. These electrodes can be the electrodes that are implanted in the cochlea or extra cochlear electrodes—any electrodes that are utilized to evoke a hearing percept can be used in some embodiments. As just noted, the electrode(s) can also be the extracochlear electrode that are used for the return in monopolar stimulation (e.g., the so-called ball electrode, or the plate electrode which is located on the receiver stimulator of the cochlear implant, etc.). These also can be additional electrodes that are added to the cochlear implant electrode array, such as electrodes arrayed as seen in FIG. 5, which are integrated with or otherwise in communication with the cochlear implant.

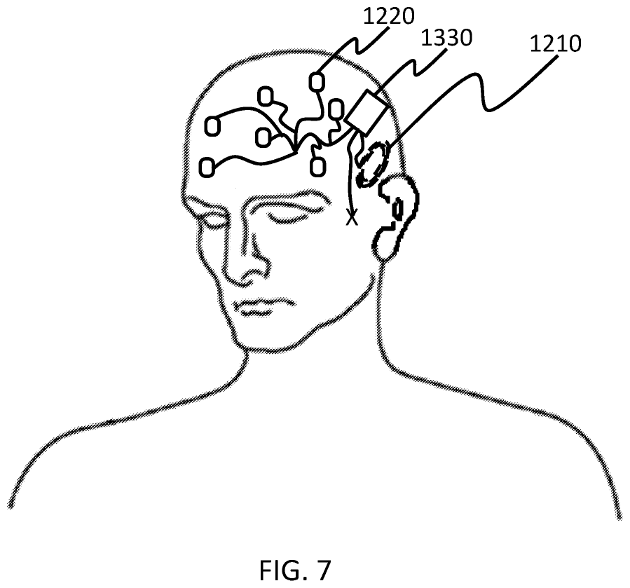

Along these lines, FIG. 7 presents an exemplary embodiment of a modified version of the embodiment of FIG. 6 detailed above. In this embodiment, which is presented in functional conceptual terms (e.g., coil 1201 and housing 1330 would be part of an integrated assembly), a cochlear implant is represented with the cochlear implant electrode array represented by the “X.” This is an example of how a cochlear implant electrode array can be integrated with an EEG read electrode apparatus. In this embodiment, the housing 1330 can include the implanted circuitry and components of a cochlear implant electrode array, which can be modified to have the functionality for the purposes of EEG reading or can instead also include in that housing separate circuitry for the purposes of EEG reading.

It is also noted that FIG. 7 conceptually represents different types of hearing prostheses than a cochlear implant. The middle ear actuator can be represented by the “X,” or the implanted actuator of a bone conduction device can be represented by the “X,” and the housing 1330 can include the circuitry that is utilized to control the implanted actuator, while also including house there in the circuitry that can enable the EEG reading.

It is also noted that in at least some exemplary embodiments, housing 1330 can include a speech processor or the like, such as that which would be implanted in the case of a totally implantable hearing prosthesis.

Returning back to the features associated with sound, as can be seen, in at least some exemplary embodiments, the prosthesis (or other medical device) can include an acoustic sensor. In an exemplary embodiment, the acoustic sensor can be an implantable/implanted acoustic sensor and/or can be an external acoustic sensor. In some embodiments, this can be a microphone or the like. In some embodiments, this implanted acoustic sensor can be utilized to obtain data associated with a sound environment of the hearing prosthesis, as noted above. It is noted that the utilization of an implanted acoustic sensor can have utilitarian value with respect to capturing body noise or the like. Indeed, in some embodiments, features captured in body noise monitoring are able to indicate the physiological state of the user. Such may indicate the level of rest or the level of stress or the stage of digestion after a meal, and may have value in determining if it is a suitable time to make a measurement. Similarly, the amplitude of body noise can have temporal characteristics or be sufficiently high so as to render the aforementioned testing less than utilitarian than that which would otherwise be the case. That is, in at least some exemplary embodiments, not only is the characteristics such as temporal fluctuations, spectral shape, or amplitude of ambient sound or the like ascertained and utilized to determine whether or not to implement testing, but also the characteristics such as temporal fluctuations, spectral shape, and amplitude of body sound is ascertained and also utilized to determine whether or not to implement testing.

Corollary to this is that in at least some exemplary embodiments, the medical device includes an implantable component configured to execute acoustic probing using the acoustic sensor.

In some embodiments, probing can be active probing. In some embodiments, the data obtained based on the active probing is non-movement data associated with the recipient, and in some embodiments, it is the physiological data, and in some embodiments, the probing is movement data. Thus, at least some of the determinations can be a determination whether or not to initiate active probing, and such determinations, in some embodiments, can be based on the non-movement data associated with a recipient of the medical device.

In some embodiments, the non-movement data is biologically based data. In some embodiments, the data (irrespective of whether it is non-movement data) can be non-biologically based data.

Note further that in an exemplary embodiment, the medical device can include only one sensor system, which sensor system can be utilized to collect both physiological data and non-physiological data. In an exemplary embodiment, the data obtained based on the active probing detailed herein is physiological data.

Note also that the teachings detailed herein are not limited to simply the utilization of sound capture devices to obtain data upon which determinations and/or analyses detailed herein are based. In this regard, in an exemplary embodiment, there is a medical device that is configured to obtain data indicative of a type and/or an amount of neuron activity of a recipient of the medical device and evaluate the obtained data. Concomitant with the other embodiments detailed herein, the device can be further configured to determine whether or not a data collection activity should be commenced based on the evaluation. With respect to the ability to obtain data indicative of a type and/or amount of neuron activity of a recipient, this can be executed utilizing the device of FIG. 6 or FIG. 7 etc. Alternatively, and/or in addition to this, the electrodes of the cochlear implant electrode array can be utilized. In some embodiments, the same electrodes can be utilized to obtain the data indicative of a type and/or an amount of neuron activity that are utilized for the data collection. In other embodiments, the electrodes are separate electrodes. Indeed, consistent with the teachings detailed above with respect to some embodiments, the medical device is not the device that executes the data collection activity (while in other embodiments, it is the device that executes the data collection activity).

Note also that it is not necessarily the case that the electrodes are utilized to obtain data indicative of the type and/or an amount of neuron activity. Any device that can enable such can be utilized. Still further, it is noted that while the embodiments disclosed herein have been directed towards implanted electrodes that are utilized to obtain data, in some embodiments, non-implanted or semi implanted electrodes can be utilized. Any device, system, and/or method that can enable the underlying teachings detailed herein can be utilized in at least some exemplary embodiments.

By way of example only and not by way of limitation, in an exemplary embodiment, electrodes can be utilized to detect whether or not the auditory portions of the brain are being stimulated. In this regard, such can be a latent variable that indicates whether or not the recipient is in a sufficiently quiet environment for the above-noted acoustic stimulation. Actually, the amount of sound in the environment is a latent variable that indicates whether or not the recipient is in a state where the above-noted acoustic stimulation can be executed in a manner that will result in utilitarian value vis-à-vis the data collected. In any event, by way of example, the electrodes of a cochlear implant electrode array might be able to be utilized to evaluate the type and/or amount of neuron activity in the recipient, which could be an indicator that the recipient is in a sound environment or an indicator that the recipient has brain functions that are occurring that are not conducive to achieving utilitarian value from any data that is collected. Again, the componentry associated with the ability to obtain data indicative of a type and/or an amount of neuron activity of a recipient can be integrated into any of the prostheses detailed herein at least some exemplary embodiments.

It is noted that some other embodiments can include obtaining data associated with the visual cortex. In this exemplary embodiment, electrodes can be utilized to acquire data associated with the activity of the visual cortex. By way of example only and not by way of limitation, read electrodes can be utilized to ascertain the level and/or number of neurons that are firing (analogous to how the auditory cortex is analyzed in at least some exemplary embodiments).

The more neurons that are firing, whether that be the auditory cortex or the visual cortex, can be an indicator of the level of stimulation that is being applied to the recipient at a given time. The medical device can be configured to analyze data obtained by the medical device that is indicative of the number of neurons that are firing, etc., or any other underlying indicia that can have utilitarian value, and based on the analysis, determine whether or not to commence data collection activities, etc.

In view of the above, it can be seen that in at least some exemplary embodiments, the medical device includes a plurality of sensor systems. By way of example, a medical device according to the teachings detailed herein can include a first sensor system and a second sensor system. The first sensor system can be a sensor system that is utilized to collect the data upon data collection commencement. This can be any of the read electrodes detailed herein, etc. The second sensor system can collect non-physiological data. By way of example, the second sensor can collect ambient sound in the environment of the recipient, consistent with the teachings detailed above. In accordance with the embodiments above, the medical device is configured to evaluate the collected non-physiological data to make the determination as to whether or not the data collection activity should be commenced. In at least some exemplary embodiments, the medical device is configured to evaluate the collected non-physiological data in the absence of any data that might be collected by the first sensor system/without regard to any data that might be collected by the first sensor system. Corollary to this is that in at least an exemplary embodiment, the second sensor system is configured to only collect non-physiological data. Conversely, in other embodiments, the second sensor system is configured to collect both. Also, in an exemplary embodiment, the medical device could utilize both sensor systems together to make the determination.

Thus, in some embodiments, there can be a medical device that includes a first sensor system and a second sensor system, the first sensor system is the sensor system used to collect the data upon data collection commencement, the second sensor system collects non-physiological data and the medical device is configured to evaluate the collected non-physiological data to make one or more of the determinations detailed herein.

While embodiments above have focused upon a second sensor system that collects sound or otherwise capture sound, in some alternate embodiments, the second sensor system can be a sensor system that collects light. By way of example only and not by way of limitation, ambient light can be a latent variable indicative of brain stimulation or the like. Accordingly, embodiments include capturing light and evaluating the amounts and/or contents of the light, to make the aforementioned determination about whether or not the data collection activity should be commenced. In an exemplary embodiment, a light sensor can be located at one the hearing prosthesis, such as on the behind the ear device, or on a button sound processor or otherwise on and off the ear device, etc. Indeed, in an exemplary embodiment, the light sensor of the portable handheld device 2401 can be utilized. In this regard, an embodiment is such that the handheld device can communicate a signal to the prosthesis or other medical device indicative of the amount of light, and the medical device can analyze that signal to make the aforementioned determinations. This can also be the case with respect to the microphone of the portable handheld device 2401.

While the embodiments above have in some instances at least focused on capturing and evaluating the amount of light present, a second sensor system, by way or example only and not by way of limitation, may capture video images or the surrounding environment, analyze these images to determine if certain features are present, and determine if a measurement should be made. These features, for instance, could be to determine the presence of other people in proximity to the user, or could be to determine a fixed physical environment.

It is noted that in some embodiments, the aforementioned medical devices detailed herein can be configured to determine and/or extrapolate a state of a recipient of the medical device and/or the environment of the medical device (and/or of the recipient—the two are not necessarily mutually eclusive), and determine whether or not the data collection activity should be commenced based on the determination of the state of the recipient. Functionality associated with determining can be achieved by, for example, monitoring brain waves of the like and determining that the recipients is, for example, sleeping. Functionality associated with extrapolating can be achieved by, for example, monitoring ambient sound for noises indicative of sleeping and/or for the absence of noise, which absence of noise, at least for a specified temporal period at a specified temporal location, is indicative of the recipient sleeping. Alternatively, the prosthesis can be configured to determine and/or extrapolate that the recipient is in a state of exercise or a state of physical activity for example. Another potential state could be the state of high levels of concentration. In some embodiments, any one or more of the states could be states where the data collection activity might result in data that is less than utilitarian relative to that which would otherwise be the case. That said, in some embodiments, there can be utilitarian value with respect to collecting data in those states because data associated with the state specifically desired. Still further, it can be a simple as trying to avoid waking the recipient utilizing a sound-based test.

In at least some exemplary embodiments, the medical device is configured to receive information indicative of a state of the recipient. In an exemplary embodiment, this can be achieved via real time input into the medical device by another device, such as the handheld device. Indeed, in an exemplary embodiment, the recipient can speak into the handheld device indicating his or her state. The recipient could activate an activation where the recipient could input his or her state, such as by pressing an icon indicating relaxed, happy, ecstatic, aggravated, tired, grumpy, sexually aroused, never wants to see another woman or man again, etc. further icons could include the ability to input the current activity of a recipient (exercising, reading, fishing, working, driving, driving in terrible traffic, etc.). Note also that instead of, or alternatively in addition to, the utilization of icons, a voice system could be utilized to receive the data. That said, the medical device can also be utilized in such a manner, bypassing the handheld device. For example, the microphone of a hearing prosthesis can be utilized, where the recipient just declares that he or she is happy, etc. Still further, note that latent variables can be used as well to extrapolate any of the aforementioned scenarios. For example, a sound capture device of the prosthesis could capture the voice of the recipient complaining about his or her boss, shouting, etc., indicating that the recipient is in a less than happy state. Repeated horn sounds could be indicative of a recipient in traffic. Sounds indicative of the description of how attractive another person is, could be indicative of the aforementioned sexual arousal (or how unattractive that person is, etc.). In any event, the medical device, one way or another, is configured to receive input indicative of one or more of the aforementioned scenarios. The medical device can be configured to receive these input(s) and evaluate the input(s) and determine whether or not the data collection activity should be commenced. Thus, embodiments include utilizing non-latent variables.

Note also that in at least some exemplary embodiments, the medical device can be configured to identify a geographic location of the recipient and/or an environment in which the recipient is in. This can be done utilizing GPS technology and/or computer-assisted locational devices such as that on a smart phone or the like, etc. This can also be done utilizing the aforementioned scenario such as sound capture (horns equating to traffic, sound of prolonged typing indicating work, etc.). Indeed, many hearing prostheses include advanced scene classification systems and algorithms. These are utilized to analyze a sound environment, and based on the analysis, adjust a hearing prosthesis to better present the sounds associated with that environment versus other settings which would be more utilitarian for other environments. Here, instead of adjusting the hearing prosthesis, the basic results of the sound environment analysis are utilized to extrapolate the environmental conditions of the recipient. Note also that visual devices can be utilized to extrapolate the environmental condition of the recipient. In this regard, advanced image processing can be utilized to determine a given location of the recipients, etc. This can be done utilizing a tiny camera located on a hearing prosthesis or on a medical device or on, for example, the portable handheld device 2401 detailed above. Thus, in some embodiments, the occurrence is a locational existence of the recipient (e.g., at an amusement park, at a football stadium, at work, etc.). Further in an exemplary embodiment, the first data is based on captured sound captured by the device of the recipient. Also, cameras can be used to collect data to evaluate other things, such as a condition of the recipient (a “selfie” can be taken, and image recognition software can make a determination if the recipient “looks himself,” and based on that determination, other determinations can be made, etc.).

In an exemplary scenario, there can be utilitarian value by determining whether or not, for example, the recipient is at an amusement park or the like. When the recipient is riding on a roller coaster, it might be less than utilitarian to collect the data. Accordingly, based on the data obtained associated with the environment of the recipient, data collection may or may not be commenced.

In an exemplary scenario, there can be utilitarian value with respect to determining tertiary environmental factors through the above analyses. For example, the utilization of geographical location as well as cameras (to determine if the person is outside or inside or in a vehicle, etc.) to determine the atmospheric pressure, the ambient temperature, the humidity, the wind characteristics, any weather characteristic, that can be utilitarian (wind, rain, sun, bright sun, night, day, etc.) and so forth at the environment where the user is located. All of this can be utilized to evaluate whether to implement measuring and/or whether to discount measurements, etc.

Accordingly, in an exemplary embodiment, the medical device includes an environmental classification system. The medical device can be configured to determine whether or not the data collection activity should be commenced based on a classification of the environment by the classification system. Other types of classification systems are used in some other embodiments, and thus in some embodiments, the medical device includes an classification system and the medical device is configured to determine whether or not the data collection activity should be commenced based on a classification of a physiological feature and/or a non-physiological feature associated with the recipient and/or environment of the recipient by the classification system.

Still with reference to method 900, consistent with the embodiments above, in an exemplary embodiment, the first data can be indicative of movement of the recipient, and the second data can be any EEG measurement that is executable and/or executed by an implanted component implanted in the recipient. In this regard, there can be utilitarian value with respect to taking EEG measurements, or otherwise evaluating EEG measurements that are taken when the recipient is stationary, as the measurements can be more indicative of the underlying phenomenon associated with the brain signals than that which would be the case of the recipient was moving. Indeed, in this regard, in some embodiments, the actions are taken when the recipient is stationary or moving and/or when the prosthesis is stationary or moving (where the noun differences are not mutually exclusive). In some embodiments, the prosthesis and/or recipient is locally stationary, which means that the prosthesis and/or recipient is not moving relative to his or her immediate surroundings (e.g., the recipient could be in an office sitting still, or could be sitting in a car that is driving on a very smooth road, but the recipient is sitting still in the car). In some embodiments, the recipient and/or the prosthesis is globally stationary, which would exclude the recipient being in a moving car, even on a smooth road, etc. In some embodiments, the occurrences that are utilized to evaluate or otherwise determine whether to execute measuring or whether to discount measuring are different from the recipient being effectively stationary. In this regard, the occurrence can be something that occurs whether or not the recipient is stationary. Alternatively, the occurrence can be delta to the recipient being stationary. Indeed, such is concomitant with the teachings herein where multiple datas can be utilized as a basis to implement or discount measuring. For example, if the accelerometer data indicates that the prosthesis is stationary, but other data indicates that the recipient is emotionally disturbed, or in a loud environment, etc., the testing may not be commenced or the data may be discounted, even though the recipient is stationary. Note also that in some embodiments, a determination of the presence of a stationary situation can be made without an accelerometer. For example, the recipient can input data that the recipient is stationary (e.g., by answering a question on the device, and selecting a yes/no prompt). Moreover, determinations can be made without sensor input regarding movement of the recipient and/or without data indicative of the movement of the recipient, and various actions herein can be taken where the recipient is stationary according to any of the scenarios herein in some embodiments. That is, even if there is no affirmative determination that the recipient is stationary and/or direct determination, the teachings herein can be executed in some embodiments.

On perhaps a more basic level, the medical device could be configured to sense a phenomenon indicative of movements of the recipient. In some embodiments, the aforementioned data collection might be less than utilitarian when collected during periods of movements of the recipient, which movement would indicate at least a modicum of physical activity. That said, in some embodiments, the phenomenon indicative of movement could be an amount of movement associated with the recipient. For example, at least some of the data collection activity could be utilitarian with respect to scenarios where the data is collected while the recipient is walking as opposed to running. Accordingly, in an exemplary embodiment, the medical device is configured to sense a phenomenon indicative of movement of the recipient, and configured to evaluate the sensed phenomenon indicative of movement to determine whether or not the data collection activity should be commenced.

It is briefly noted that at least some exemplary embodiments are such that any one or more or all of the method actions and/or functionalities detailed herein are executed by a medical device. In an exemplary embodiment, one or more or all of the method actions and/or functionalities detailed herein are executed by a prosthesis in general, such as a hearing prosthesis or a retinal prosthesis, in particular. That said, in some embodiments, one or more of the method actions detailed and/or the functionalities detailed herein can be executed by a nonmedical device under a non-prosthetic device, where data indicative of that method action or the functionalities, or the results thereof, is transferred to the medical device, so that other method actions in functionalities which depend upon such data can be executed.

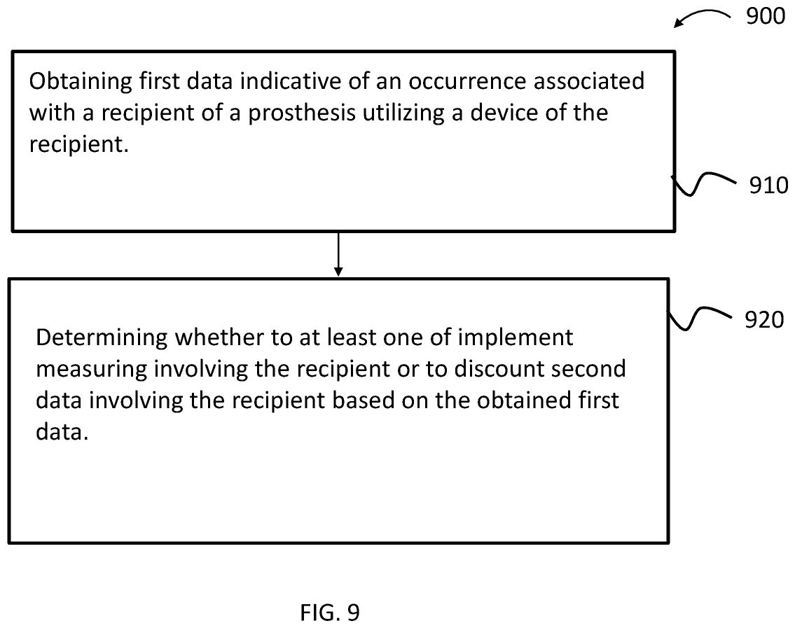

FIG. 9 presents an exemplary flowchart for an exemplary method, method 900, which includes method action 910, which includes the action of obtaining first data indicative of an occurrence associated with a recipient of a prosthesis utilizing a device of the recipient. The device of a recipient can be the prosthetic device that the recipient has received. The device of a recipient can be an implantable prosthetic device or an external prosthetic device. Further, the device of the recipient need not necessarily be a prosthetic device. Instead, it could be some form of medical device of the recipient. Moreover, in an exemplary embodiment, the device of the recipient might not even be a medical device per se. By way of example only and not by way of limitation, it could be the portable handheld electronics device 2401 detailed above. More on this below.

Method 900 also includes method action 920, which includes determining whether to at least one of implement measuring involving the recipient or to discount second data involving the recipient based on the obtained first data. In an exemplary embodiment, by way of example only, the first data is indicative of at least one of an environment of the recipient, an activity engaged in by the recipient or a state of the recipient. Consistent with the teachings detailed above, the environment can be a noise environment, a stimulating environment (amusement park, etc.), a car environment, a traffic environment, a room full of children environment, etc. Also consistent with the teachings detailed above, the activity engaged in by the recipient could be exercise, driving, reading, sleeping, etc. The state of the recipient could be aggravated or happy or ecstatic or excited, etc.

The obtained first data can be obtained by any of the devices herein, such as any of the prostheses herein or medical devices herein or the remote device such as the portable handheld device, etc. Alternatively, and/or in addition to this, the action of determining, method action 920 can be executed remotely from the device of the recipient, such as via a geographically remote server or the like. That said, method action 920 can be executed remotely from the device of the recipient utilizing another device, such as the hearing prosthesis, where the device that was utilized to execute method 910 could be a dedicated EEG monitoring device as noted above. Still further, in an exemplary embodiment, the device that is utilized to execute method action 920 can be the portable handheld device 2401 detailed above, while the device that is utilized to execute method action 910 can be the prosthesis, such as the hearing prosthesis, or any other medical device, and thus the one devices remote from the other device. Conversely, the action of determining, method action 920, can be executed by the device that is the subject of method 910/that was utilized to obtain the first data. Again, such can be implemented utilizing any of the integrated devices detailed herein and/or variations thereof.

In view of the above, the measuring of method action 920 includes utilizing and implanted devices implanted in the recipient to measure physiological feature(s) of the recipient. In this regard, in an exemplary embodiment where the second data that is obtained is also obtained by implementing measuring, both measurements associated with the possible permutations of method action 920 include utilizing implanted devices implanted in the recipient to measure physiological features of the recipient, at least in some exemplary embodiments.

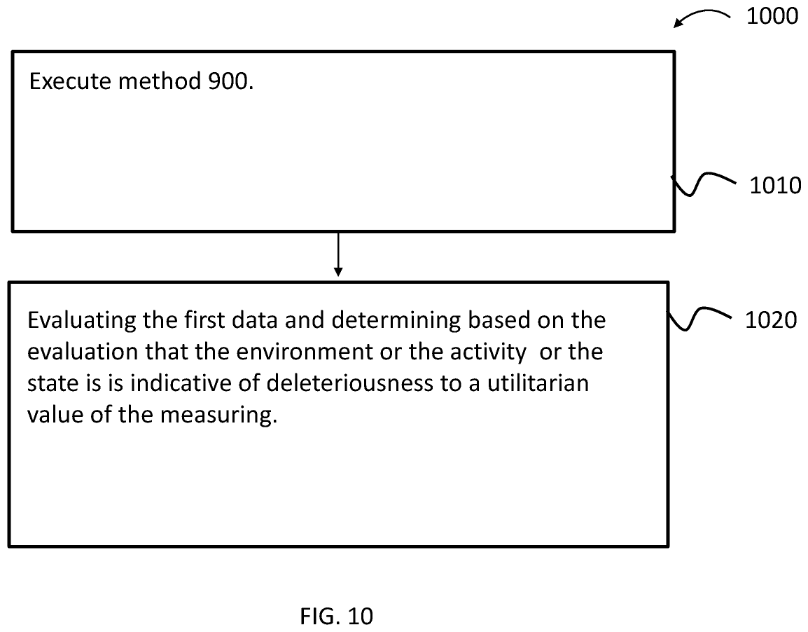

FIG. 10 presents an exemplary flowchart for an exemplary method, method 1000 includes method action 1010, which includes executing method 900. Method 1000 also includes method action 1020, which includes evaluating the first data and determining based on the evaluation that the environment, the activity and/or the state is indicative of deleteriousness to a utilitarian value of the measuring. Again, by way of example, the environment could be a noisy environment in the scenario where sound will be utilized to stimulate brain activity. The activity could be a recipient who is exercising more is at an amusement park or the like, where the activity is causing the recipient's brain to function in a manner that the recorded EEG signals will not be utilitarian because of the overall stimulus. In an exemplary embodiment, such as where a man is thinking about a woman he or she finds attractive, the state of the recipient would be arousal, and it could be possible that brain wave patterns of men in such states are suppressed, heightened, or skewed a certain way, just possibly. In an exemplary embodiment, the action of determining in method action 1020 can include discounting the second data based on the evaluation. Again, in an exemplary embodiment where there is a noisy environment, and testing was executed utilizing a hearing percept evoked by the prosthesis for example, because the recipient may not react to the hearing percept evoked by the prostheses and/or that the hearing percept evoked by the prostheses is overwhelmed by the environmental noise, the data that is achieved would be skewed or potentially not useful. Indeed, if the recipient could not perceive the sound, the data that is obtained would be like any other data that would be obtained in the absence of the sound, all other things being equal.

Briefly, with respect to discounting the second data, scenarios exist where the first data is collected irrespective of the states where the activity or the environment of the recipient. In this regard, there are some types of sensory systems that have access to sufficient power and/or processing capabilities and/or data collection working elements that the systems can collect the data on an ongoing basis, potentially continuous or semi-continuous. In at least some exemplary embodiments, this provides utilitarian value under the premise that more data is better than less data. However, there is the contrary concept that more data that includes more bad data is less utilitarian than less data that includes less bad data. Of course, there is utilitarian value with respect to more data that includes more good data and less bad data. In view of this, it can be seen that the teachings detailed herein can be utilized to achieve any of the combinations that would be desired. With respect to discounting, the concept is that you have lots of data, which includes lots of bad data, because the system that is collecting the second data is collecting it at a rate or otherwise in a manner that is ambivalent to whether or not the data that is being collected is good or bad. However, utilizing the power of the teachings according to the present application, the data that is collected can be correlated in some manner or another with the first data (temporally, numerically, etc.), and then later on (or in real time), evaluation to be made whether the first data warrants discounting of the second data. For example, if data is collected while the recipient is distracted emotionally for a various number of reasons, this second data might warrant the discounting of the first data. This as contrasted to a situation where the system or what have you determines that the second data indicates that the first data should not be collected in the first instance.

In view of the above, it can be seen that the options for implementing the teachings detailed herein are vast and expansive. The innovations according to this application enable various options for the healthcare world that heretofore simply did not exist, at least not in a practical matter for implementation.

To be clear, an exemplary embodiment of discounting could include simply ignoring various amounts of first data based on the second data. An exemplary embodiment of discounting could be deleting various amounts of first data based on the second data. An exemplary embodiment of discounting could be to engage in further actions to validate or otherwise further analyze the first data, which otherwise would not occur in other situations with respect to the second data. By way of example only and not by way of limitation, in a situation where the recipient was very excited, the first data collected during that period could indicate that there is a more likely potential for a seizure or the like because of brain activity, but because the method/system “knows” that this data was collected in a period of excitement, it might not automatically issue a warning to the recipient that a seizure was imminent (or to some other caregiver), but instead increase the frequency of monitoring and/or the length of monitoring and/or increase the extent or length, etc. This as opposed to first data indicating that a seizure might be imminent, where the second data indicated that the recipient was relaxed. In such a scenario, the first data would not be discounted, any warning might be automatically issued without waiting or the like. That is, the first data is not discounted in any way. In this regard, the concept of discounting can be a relative concept to the treatment of the data and other times or under other collection regimes.

As will be understood from the above, at least some embodiments are implemented utilizing hearing prostheses such as cochlear implants. In this regard, the cochlear implants evoke a hearing percept based on captured ambient sound. Thus, a noisy environment will result in noise being perceived by the recipient, and electrical artefacts from electrical stimulation of the cochlea. In some embodiments, the medical device can be configured so as to stop the supply of ambient environment noise to the recipient so that no sound is perceived and no electrical artefacts are recorded in the signal measurement in system 1. In another embodiment, the background noise representation is stopped, and then an electrical probe is activated, which is the sound that is utilized for the test. This can enable at least some testing even in an environment that would otherwise be deleterious to the obtained data. Accordingly, in an exemplary embodiment, in a variation of the method actions detailed herein, upon the analysis of the obtained first data, a determination can be made to implement measuring involving the recipient, but in a controlled manner where the sound of the ambient environment is blocked off by the prostheses. That is, method action 920 could come with a modification that further includes preventing the evocation of a hearing percept based on ambient sound during the test. Instead, such as, for example, in the case where an acoustic probe is used, the only hearing percept that is evoked is based on the test sound.

In an exemplary embodiment, the data that is the subject of the methods herein is non-EEG data and/or non-EKG data.

In an exemplary embodiment, the medical device could be configured to notify the recipient that there will be a period of time where the recipient cannot hear the ambient environment. Thus, the recipient will be warned. In an exemplary embodiment, medical device can be configured to ask or otherwise request input from the recipient as to whether or not such an occurrence would be okay with the recipient. The medical device can be design, in some embodiments, to require an affirmative input by the recipient to proceed, while in other embodiments, the medical device would not proceed only in the case where the recipient overrides the medical device.

Method 900 can have utilitarian value with respect to determining whether or not to step up measuring or recording or the like from a baseline recording/measuring regime to something more sophisticated or otherwise more processor intensive or otherwise data intensive. In this regard, data logging features of a given medical device or a hearing prosthesis can periodically obtain the first data at a given rate and/or in given amounts which are smaller than that which would be the case during a period of greater interest or otherwise during an event associated with the recipient, such as an epileptic seizure or a pre-epileptic seizure period. Thus, in an exemplary embodiment of method 900, a scenario can exist where the method includes executing a low fidelity recording, and then upon a determination to implement measuring involving the recipient, the medical device implements high fidelity recording. Indeed, in an exemplary embodiment, even in the absence of determining to implement measuring, a low fidelity recording can already have taken place upon the action of determining. In this regard, method action 920 can entail determining to change from low fidelity recording to high fidelity recording. Method action 920 can also entail determining not to change from low fidelity recording to high fidelity recording and just maintain the low fidelity recording efforts.

Thus, in an exemplary embodiment of method 900, the measuring is a high fidelity recording and low fidelity recording is occurring at the time of the determination. The action of determining, method action 920, includes determining to implement the high fidelity measuring, and thus transition from low fidelity measuring to high fidelity measuring.

Still with reference to method 900, the obtaining action, method action 910, is executed utilizing hearing prostheses components. This is distinguished from utilizing a hearing prosthesis. In this regard, there are devices and systems that are developed and otherwise made from components that are cannibalized from hearing prostheses designs. In this regard, there are devices that correspond to, for example, cochlear implants, which are configured to execute method 900 and the variations thereof. Still further, there are devices that are not cochlear implants per se, but utilize components from cochlear implants or other implants/prostheses, such as FDA approved/licensed products, such as FDA approved/licensed cochlear implants as of Sep. 1, 2018.

For example, microphone components and sound capture devices and/or scene classification algorithms and/or sound level detection/identification devices, etc., can be utilized in a device that is not a hearing prosthesis per se. Instead, these devices can be utilized to obtain the first data and/or analyze the first data. Still further, in an exemplary embodiment, simultaneous with the action of obtaining first data, and/or contemporaneous with the action of obtaining first data, the components that are utilized to obtain the first data and/or analyze the first data are also used elsewhere for hearing prostheses purposes, such as to ultimately evoke a hearing percept utilizing a hearing prosthesis. In this regard, embodiments include utilizing components that are utilized in hearing prostheses for non-hearing prostheses purposes. Thus, there are methods that include utilizing those components in a hearing prosthesis at the same time the design for those components or otherwise components having the exact same given features and/or parts and/or structure, etc., are utilized in a non-hearing prostheses device to implement at least some of the teachings detailed herein.

Note also that noise cancellation techniques can be utilized to ascertain ambient noise/environment noise. For example, many hearing prostheses can include noise cancellation devices. The operation of those noise cancellation devices can be analyzed or otherwise evaluated to determine the amount of noise in the ambient environment. Again, teachings detailed herein can be directed towards the utilization of FDA approved componentry of hearing prostheses devices for non-hearing percept evoking purposes, but instead to ascertain or otherwise identify the occurrence of other events. To be clear, the aforementioned body noise system or the noise cancellation system may never be utilized to evoke a hearing percept. That is, concomitant with the teachings detailed above, there are devices systems and methods that include hearing prostheses components and/or methods that are utilized with hearing prostheses that would otherwise be utilized to evoke a hearing percept where no hearing percept is evoked based on the use thereof.

Corollary to the above is that in at least some exemplary embodiments, the devices systems and/or methods disclosed herein relating to hearing prostheses technology and/or retinal implant/bionic eye technology, are utilized with people that have no hearing impairments and/or vision impairments or otherwise have no sensory impairments. In an exemplary embodiment, the devices systems and/or methods disclosed herein relating to hearing prostheses technology and/or retinal implant/bionic eye any one or more of the aforementioned senses under the Americans with Disabilities Act, as of Sep. 15, 2018, as the regulations and laws have been interpreted on this date. That is, the person that is associated with the methods and devices herein is a person who would not be considered to have a given sensory disability under the law. That is, the person would not be considered to have a vision disability or a hearing disability as a matter of U.S. law under that act. This is not to say that the person might not be disabled under that act were that the person might not having other sensory disability. This is to say that a specific sensory disability is not existing with respect to a given person.

This is also not to say that disabled persons cannot avail themselves of the teachings detailed herein. This is to say that the teachings detailed herein can be 100% applicable and overrides utilized with people who have perfectly fine senses.

In an exemplary embodiment, the teachings detailed herein are executed on persons or otherwise in relation to persons who are not legally deaf and/or are not legally blind under the laws of the U.S. and/or under the laws of the State of California as exists and has been interpreted as of Sep. 15, 2018.

In an exemplary embodiment, the teachings detailed herein are executed on persons or otherwise in relations to persons who can hear sounds at 500, 750, 1000, 1250, 1500, 1750, 2000, 2500, 3000, 3500, 4000, 5000, 6000, 7000, 8000, 9000, 10,000, 12,500, 15,000, 16,000, 17,000, 18,000 and/or 19,000 Hz in a manner that corresponds to that which a 50 percentile human factors engineering person would be considered to hear such as of Sep. 15, 2018, which person is a 20, 30, 40, 50, 60 and/or 70 year old person (the human factors person, not the person that is the subject of the method) who is a resident of the United States of America and/or a natural born citizen residing in the United States of America on Sep. 15, 2018, of any race or of any sex or of a male sex or a female sex (US military manual on human factors engineering can be applicable in some embodiments, again as that manual exists on the Sep. 15, 2018, date).

Note further, the persons that are the subjects of the methods and/or those that utilize the devices can be persons who have hearing and/or sight capabilities such that Aetna or Blue Cross-Blue Shield Personal Choice or the healthcare system used at the Boeing Company for their Philadelphia plant for the greatest number of their non-exempt employees would not reimburse a person for a hearing prostheses because there are hearing is not bad, as of how those policies would be implemented as of Sep. 15, 2018, in the state of California or the Commonwealth of Pennsylvania.

Referring back to the external device of FIG. 4, that device can be utilized in combination with the exemplary EEG systems and/or EKG systems disclosed herein. Indeed, in an exemplary embodiment where, for example, the implanted coil of the EKG system detailed herein is located in the upper reaches of the torso, such as at the top of the chest, it is possible to utilize the external device 1440 with such a system by snaking the lead 1472 downward through a person's shirt collar or the like to the person's chest or shoulder. That said, in alternate embodiments, a specialized external device especially for the EKG system can be utilized, where, for example, the non-coil portions (e.g., the equivalent of the BTE component 1426) is worn on a chain around the person's neck like a pendant, and the coil is magnetically adhered to the coil inside the person. Further, an off-the-ear (OTE) device could be used, which can be a single unit located over the coil, wherever such is located. This device would not be on a pendant, but instead could be held by a magnet, etc., to the recipient.

Again, in some embodiments, the external device is basically an external device of a hearing prostheses, whether that be a cochlear implant, a middle ear implant, a bone conduction device, or a conventional hearing aid. In some embodiments, this external device is utilized for a person that does not have hearing problems in accordance with the above. Still further, in at least some embodiments, this external device is utilized in a manner that does not include evoking a hearing percept or a sight percept or a sensory percept, etc. utilizing the device per se, with the possible exception of utilizing the device for testing purposes.

Embodiments include utilizing a hearing prosthesis or a hearing prosthesis-based component in a manner that does not evoke a hearing percept, at least for a day or two or more, if not forever, at least not for purposes unrelated to measuring. Such is also the case with respect to other types of sensory prostheses, such as a light prosthesis, the so-called bionic eye. In this regard, in an exemplary embodiment, there are methods that include utilizing a device that has a sound processor or otherwise is configured for sound processing, which processing could be utilized to evoke a hearing percept if the device was utilized in a hearing prosthesis. There are also methods that include utilizing a device that has sound processor technology, such as noise cancellation and/or body noise cancellation or detection features, etc., which could be used to do so if such was utilized in the hearing prostheses. In these methods, the devices are utilized during periods lasting at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 200, 250, 300, 350, 400, 500, 600, 700, 800, 900 or 1000 hours or days without being utilized to evoke a hearing percept or a sight percept or otherwise a sensory percept, again with the exception of doing so to obtain measurements. Accordingly, there can be methods that include performing any one or more of the actions detailed herein with the basic processing components if not all of the components of a hearing prostheses, with or without output components (receiver/speaker, actuator, electrodes), to do things unrelated to evoking a hearing percept. Such can also be the case with a retinal implant (which may or may not have the electrodes), where things are done/the device is utilized to do things unrelated to evoking a light percept.

In an exemplary embodiment, 30, 35, 40, 45, 50, 55, 60, 65, 70, 75, 80, 85, 90, 95 or 100% of the components of the devices according to some embodiments, on cost basis for a given point in time that enables apples to apples comparison, are components that would be found in a hearing prostheses manufactured on that date by at least one hearing prostheses manufacturing company in the world. In some exemplary embodiments, the aforementioned percentages are for the components that are related to one or more of determining whether or not a data collection activity should be commenced and/or obtaining the data that is utilized to determine such, and/or whether or not data collected should be discounted and/or obtaining the data that is utilized to determine such, and/or the components that are for obtaining the aforementioned first data indicative of an occurrence associated with a recipient and/or to determine whether to at least one of implement measuring or to discount measuring, and/or the components of the second sub-system and/or the third sub-system. In an exemplary embodiment, the above noted percentages exclude software, while in other embodiments, the above noted percentages include the software.

Consistent with the above, in at least some exemplary embodiments, the obtaining action, action 910, and/or any analysis associated with the data that is obtained by that action, can be executed utilizing cochlear implant totally implantable hearing prostheses implant components. Again, the idea is to cannibalize or otherwise utilize a given design that exists in otherwise was developed for a hearing prosthesis for non-hearing prostheses purposes.

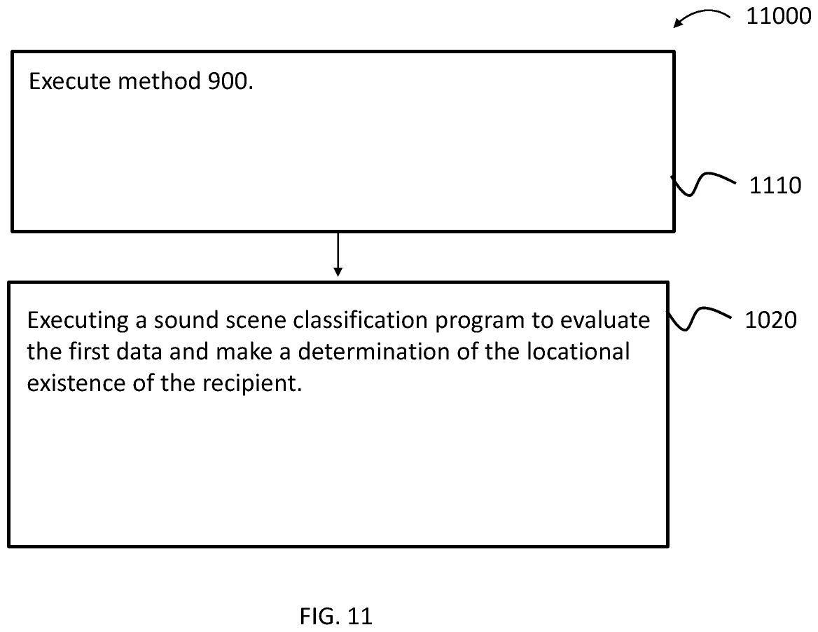

As noted above, there can be utilitarian value with respect utilizing sound scene classification and the like with respect to evaluating the first data of method action 910. Accordingly, FIG. 11 presents an exemplary flowchart for an exemplary method, method 1100, with includes method action 1110, which entails executing method 900. Method 1100 also includes method action 1120, which entails executing a sound scene classification program to evaluate the first data and make a determination of the locational existence of the recipient. Thus, method action 1120 occurs in between method action 910 and method action 920 in at least some exemplary embodiments. In this regard, it is noted that any method action detailed herein can be executed in any order relative to any other method actions detailed herein providing that the art enable such illness otherwise identified. Accordingly, the order of presentation of given method actions detailed herein does not necessarily correspond to the actual order in which those method action will be executed. That said, in other embodiments, such is the order in which those method actions will be executed.

Sound scene classification is a technology that has been developed and otherwise perfected by the Cochlear Limited company of Sydney, Australia. Sound scene classification can be utilized as a latent variable in some instances to determine any number of things, such as location, the activity in which the recipient is participating, or even a state of the recipient. By way of example only and not by way of limitation, a sound scene constituted of loud rock music or political commentary television might be a latent variable that indicates that the recipient might be more edgy, as compared to listening to elevator music, listening to a weather report or in silence. In an exemplary embodiment, there is utilization of sound scene classification systems as disclosed in U.S. Patent Application Publication No. 2017 0359659, filed on Jun. 9, 2016, to inventor Alex Von Brasch, of Australia, entitled Advanced Scene Classification For Prosthesis.

In an exemplary embodiment of method 900, the obtained first data in method action 910 is obtained at a plurality of times over a first temporal period. By way of example only and not by way of limitation, the first data can be collected every, less than, or more than X seconds or minutes, where X equals 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 5.5, 6, 6.5, 7, 7.5 8, 9, 10, 11, 12, 13, 14, 15, 16, 17, 18, 19, 20, 25, 30, 35, 40, 45, 50, 55, 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900, 1000, 1100, 1200, 1300, 1400, 1500, 1600, 1700, 1800, 1900, 2000, 2100, 2200, 2300, 2400, 2500, 3000, 4000, 5000, 6000, 7000, 8000, 9000, 10000, 15000, 20 k, 30 k, 40 k, 50 k, 60 k, 70 k, 80 k, 86400, 90 k, 100 k, 125 k, 150 k, 175 k, 200 k, 250 k, 300 k or more or any value or range of values therebetween in 0.01 second increments (e.g., 0.25, 22.3, 5 to 30.22 seconds, etc.).

It is further noted that the first data can be collected at nine even intervals, such as in some instances, every five seconds and in other instances every 10 seconds, etc. is also noted that in some embodiments, consistent with the teachings detailed above, the data collection can be suspended for any of the aforementioned temporal periods owing to the various scenarios that could occur that might indicate that data collection would result in data that includes less utilitarian value than that which would be collected another temporal periods. Note also that in some embodiments, depending on the scenario, data collection could be increased to a rate falling with any of the aforementioned values are variations thereof.

In any event, the obtained first data is obtained at a plurality of times over a first period. Note also that the first temporal period can correspond to any of the values of X seconds or minutes detailed above, which includes any range of values therebetween.

Note also that the plurality of times can correspond to more than, less than or equal to Y times, where Y is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 110, 120, 130, 140, 150, 160, 170, 180, 190, 200, 210, 220, 230, 240, 250, 275, 300, 350, 400, 450, 500, 550, 600, 650, 700, 800, 900, 1000, 1250, 1500, 1750, 2000, 2500, 3000, 3500, 4000, 4500, 5000, 6000, 7000, 8000, 9000, 10 k, 11 k, 12 k, 13 k, 14 k or 15 k or more or any value or range of values therebetween in integer increments.

Still further, in an exemplary embodiment, the action of determining in method action 920 is respectively executed a plurality of times for the respective obtained first data. The plurality of times can equal to any value of Y detailed above, or any value of Y minus Z, where Z equals 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 110, 120, 130, 140, 150, 160, 170, 180, 190, 200, 210, 220, 230, 240, 250, 275, 300, 350, 400, 450, 500, 550, 600, 650, 700, 800, 900, 1000, 1250, 1500, 1750, 2000, 2500, 3000, 3500, 4000, 4500, 5000, 6000, 7000, 8000, 9000, 10 k, 11 k, 12 k, 13 k, 14 k or 15 k or any value or range of values therebetween in integer increments.

Also, in some embodiments, the obtained first data includes a respective data indicative of a sensorially noisy environment. A sensorially noisy environment is not limited to a sound noisy environment. This could be due to visual stimulation as well. Indeed, visual noise, as opposed to sound noise, can be a feature associated with bionic eyes and retinal implants, etc. to be clear, the phrase “noise” and the phrase “noisy” as used herein, without modifiers, always corresponds to sound noise. The phrase “sensory noise” and variations thereof is a genus that encompasses the various species, by way of example, of visual noise and sound noise. Thus, in an exemplary embodiment, the data indicative of a sensorially noisy environment can be, in some embodiments, the data indicative of a sound noisy environment. To be clear, consider visual noise, which can be also harmful or otherwise deleterious to accurate measurements or otherwise can influence or otherwise skew the measurements. Any type of sensory stimulation that is an equivalent to sound noise for the purposes of impacting the measurements that are taken in the embodiments herein can result in a sensorially noisy environment.

Indeed, this raises another point. One utilitarian value of the teachings detailed herein is to identify situations where the recipient or person is experiencing “sensory overload.” Sensory overload, at least in some exemplary embodiments, could be, in at least some exemplary embodiments, the worst situation with respect to the measurements that are obtained, because at least in some embodiments, this would most skew the data, and thus be most likely to result in false positives or false negatives, which is in part, at least a goal of some embodiments of the teachings detailed herein (identifying false positives and/or false negatives and/or identifying false concerning data and/or false unconcerning data and/or avoiding false positives and/or false negatives, or more accurately, avoiding the collection of data that includes data that would result in a false positive and/or a false negative and/or data that would include false concerning data and/or false on concerning data, etc.—the teachings detailed herein can in some embodiments, be utilized to do any one or more of the aforementioned things). Accordingly, embodiments also include identifying a sensorially overloading environment with respect to the action associated with obtaining the first data, and proceeding accordingly based on such an identification or lack of an identification.

Further, a respective determining of action 920 includes respectively determining to not implement measuring temporally correlated to the respective obtained first data and/or to discount the respective second data temporally correlated to the respective obtained first data. Moreover, in some embodiments, the obtained first data includes a respective data indicative that the noisy environment is no longer present and respective determining includes determining to implement the measuring temporally correlated to the respective obtained first data or to not discount the second data temporally correlated to the respective obtained first data.

Further, with respect to the above, in an exemplary embodiment, the obtained first data includes first sub-data obtained during a first temporal period when the recipient is experiencing a first classification of occurrence associated with the recipient. Also, the obtained first data can include second sub-data obtained during a second temporal period after the first temporal period when the recipient is experiencing a second classification of occurrence associated with the recipient. In an embodiment where one the above apply, the action of determining, action 920, includes a first determining to implement measuring temporally correlated to the first sub-data or to not discount third sub-data included in the second data and second determining to halt measuring temporally correlated to the second sub-data or to discount fourth-sub-data included in the second data.

For example, where the occurrence is movement of the recipient, the first classification of occurrence can be non-significant movement of the recipient and the second classification of occurrence can be the occurrence of significant movement of the recipient. Thus, in embodiments where the occurrence of significant movement of the recipient is considered to be something that would do it seriously affect measurements of the like, the action of determining in method action 920 can include a first determining to implement measuring temporally correlated to the first sub-data. This because the classification of the occurrence is non-significant movement of the recipient. Hence, the measurements are likely to be good measurements or otherwise measurements having utilitarian value, and the measurements should proceed to be taken. With respect to embodiments where measurements are taken irrespective of the occurrence associated with a recipient of the prosthesis, where instead the measurements are discounted, the determining action 920 could include not discounting third sub-data included in the second data. For example, the second data could be EEG measurements which are taken during the first temporal period and the second temporal period and periods before and/or after. The EEG measurements taken during the first temporal period would thus correspond to third sub-data and thus would not be discounted because of the classification.

Still further, the determining action in action 920 can also include the above-noted second determining, which is a determination to halt measuring temporally correlated to the second sub-data. This because the second classification of the occurrence is significant movement of the recipient. Hence, the measurements are likely to be less than good measurements or otherwise measurements having a value that is less than utilitarian, at least compared to measurements that would be obtained when the recipient is not moving. With respect to embodiments where measurements are taken irrespective of the occurrence associated with a recipient of the prosthesis, where instead the measurements are discounted, the determining action 920 could include discounting fourth sub-data included in the second data. For example, the second data could be EEG measurements which are taken during the first temporal period and the second temporal period and periods before and/or after etc. The EEG measurements taken during the second temporal period would thus corresponds to fourth sub-data and thus would be discounted because of the classification.

It is noted that while the above exemplary embodiment has been described in terms of nonsignificant movement and significant movement, other embodiments include other scenarios, such as non-significant distraction and significant distraction, non-significant excitement and significant excitement (this can be expanded to whatever species of the genus excitement that could be utilitarian), non-significant irritation and significant irritation, non-significantly fatigued and significantly fatigued, etc. Note further that the occurrence associated with the recipient could be according to various other aspects disclosed herein, such as the recipient being at work, being at an amusement park, driving, driving in traffic, relaxing, sleeping, etc. Thus, the classifications could be grouped into two or more groups (more on the “more” below), one group being classified as something that is conducive to obtaining good measurements and the other group that is something that is not conducive to obtaining good measurements, and the above method would be implemented accordingly with the appropriate modifications.

With respect to the “more” in the phrase “two or more groups,” there could be a third classification or a fourth calcification, etc. A third classification could be “in the middle.” This could trigger the beginning of collection of data which would then be considered for discounting at a later time. This in a regime where the method has triggers to begin collecting data or not collect data depending on the first and second classifications respectively. A third and fourth classification could be both “in the middle,” where the third classification results in not discounting the data if other features are present and the fourth classification results in discounting the data if other features are present.

The above raises another point. It is noted that the beginning of collection of data is not mutually exclusive with the action of discounting the data. In other words, the actions detailed above can exist separately and individually. For example, a method could include beginning the collecting of data and then subsequently discounting that data. A method could include beginning collecting of data and not discounting some of that data and discounting other parts of that data. A method could include always collecting data, and discounting some of the data and not others, etc. Any permutation that will provide utilitarian value can be utilized in at least some exemplary embodiments.

Referring back to embodiments where sound is utilized to evoke a brain response, or even in embodiments where sound is not used to do so, but simply where noise can skew the measurements, in an exemplary embodiment, the first classification is non-significant exposure of noise (sound noise, consistent with the convention detailed above where the use of noise without a modifier corresponds to sound noise) to the recipient and the second classification of occurrence is the occurrence of significant exposure of noise to the recipient. Also, as will be understood from the above, some embodiments can include the utilization of visual stimulus to evoke a brain response, or even in embodiments where visual stimulus, such as light, is not utilized to do so, but simply where light and/or visual stimulus can skew the measurements, in an exemplary embodiments, where the occurrence is exposure of the recipient to visual noise, the first classification is non-significant exposure of visual noise to the recipient and the second classification of occurrence is the occurrence of significant exposure of visual noise to the recipient. Any other type of sensory noise that can impact measurements can be taken into account as well. Smell could be one. Tactile input could be another. Electromagnetic fields could be a further one. Temperature could be a subsequent one. Wind can also be another one. further subsequent one. Air pressure could be a subsequent further one. Taste as well. With regard to the latter, latent variables might be utilized to determine or otherwise ascertain whether or not the recipient is eating or chewing or the like. In this regard, body noise algorithms could be utilized, instead of and/or in addition to its typical operation to remove body noise from the signal in this case to remove non-body noise from the signal, to obtain data indicative of such actions, and deduce or otherwise infer, by treating the data as latent variable, that the recipient is eating or drinking or what have you. In such exemplary embodiments, the teachings detailed herein could be such that measurements are not taken while the recipient is eating or chewing or drinking, or the data is discounted accordingly. The opposite could be the case as well. Accordingly, exemplary embodiments include devices, such as medical devices, such as prostheses, that are configured to identify or detect the occurrence of body noise, otherwise evaluate a body noise, and use that as a basis to engage or not engage measuring or to discount or not discount measurements, etc. Thus, embodiments include prostheses that are configured to detect body noise. In an exemplary embodiment, the body noise cancellation algorithms can be analyzed during operation thereof to determine the amount of body noise that is present or the type of body noise, etc. Accordingly, embodiments include modified hearing prostheses where the underlying processor or circuitry, etc., that is utilized for body noise cancellation is also utilized to extract data for the purposes of determining the occurrence of things that might impact taste. For example, the amount of cancellation in given frequencies could be indicative of body noise. Noise that is detected and certain frequencies by an implanted accelerometer or the like can be utilized to identify the occurrence of such body noises. This can also be the case with respect to the amplitude etc. Any device, system, and/or method that can utilize body noise detection and/or cancellation techniques in the prior art as modified to implement the teachings detailed herein to ascertain whether or not the recipient is eating or drinking or chewing or smoking or the like can be utilized in at least some exemplary embodiments.

In an exemplary embodiment, the devices detailed herein can be configured to identify the existence and/or the absence of at least one of internal body noise (e.g., using any of the body noise cancellation and/or noise detection devices, systems and/or methods available as of Oct. 31, 2018 on the market in the United States and/or approved by the FDA), scalp EMG, eye EMG, eye movement, body temperature, body heart rate, body blood pressure or user speech that at least one of meets or does not meet a predetermined criteria, and determine whether or not the data collection activity should be commenced based on the identified existence and/or absence.

In some embodiments, again where the occurrence is a locational existence of the recipient, the first data is based on sound captured by the device of the recipient. Again, in an exemplary embodiment, scene classification can be used in order to identify the locational existence.

Embodiments include systems that can have utilitarian value. For example, now with reference to FIG. 12, there can be a system 1210 comprising a first sub-system 1220 configured to sense a phenomenon associated with an individual, a second sub-system 1230 configured to at least one of capture sound, capture light, or capture electromagnetic radiation, and a third sub-system 1240 configured to at least one of (i) analyze output from at least the second sub-system and determine at least one of whether to activate the first sub-system or a level of activation of the second sub-system or (ii) analyze output from at least the second sub-system and the first sub-system and determine at least one of whether to activate or a level of activation of a fourth sub-system that stimulates the recipient. FIG. 12 presents the system 1210 with the various subsystems enclosed in dashed lines. This is because in some embodiments, system 1210 is a single integrated device that includes all three of the subsystems, while in other embodiments, the subsystems are separate devices and/or two of the subsystems are in a device that is separate from that of the third subsystem, or even the various subsystems could have sub subsystems that are spread out over multiple devices, and some devices can include sub subsystems from different systems in some embodiments. An exemplary embodiment that utilizes captured electromagnetic radiation can be a device that can tell whether the external coil is being used, where the presence or absence of such could be determinative as to whether or not to implement testing and/or whether or not to discount test results, etc. In another embodiment, such can be indicative of location of the recipient, which also can be used to make the various determinations detailed herein, at least in part.

To be clear, referring back to FIGS. 3A and 3B, it can be seen that in at least some exemplary embodiments, any one or more of the functionalities detailed herein can be executed by the prostheses and/or by the handheld electronics device, and/or remotely via communication with the tele-coil or the like. In this regard, in an exemplary embodiment, the various determinations and/or detections that can be deduced from the measurements of the like, can be executed by a separate component from that which took the measurements and/or from that which determined that the measurement should be taken or otherwise not discounted, etc. Any method action disclosed herein, and/or any functionality disclosed herein can be executed by any one or more of the devices disclosed herein unless otherwise noted, providing that the art enables such.

Consistent with the teachings detailed above, in an exemplary embodiment, the first subsystem is an EEG monitor. Again, this can be a standalone subsystem as detailed above, or can be integrated with other medical device systems/subsystems, such as hearing prostheses systems/subsystems. In an exemplary embodiment, as will be described below, subsystem can be an EKG monitor. Any device that can have utilitarian value with respect to monitoring a phenomenon associated with the recipient can be utilized in at least some embodiments.

In at least some embodiments, system 1210 is configured to analyze at least the output from at least the second sub-system and identify at least one of a locational situation of the recipient, an activity in which the recipient is engaged, or a state of the recipient, again, concomitant with the various teachings herein. The system 210 is further configured to make the determination(s) based on the identification. Again, the idea is that there are situations that are more utilitarian than others with respect to collecting data and/or obtaining data that is more utilitarian than other data collected at other times, and the teachings detailed herein can enable the identification of a given situation, or at least provide indicators that one situation exist versus another situation, etc.

In an exemplary embodiment of the system of FIG. 12, the system is configured to analyze at least the output from at least the second sub-system and identify at least one of a locational situation of the recipient or an activity in which the recipient is engaged or a state of the recipient. Further, the system can be configured to make the determination(s) based on the identification. In this regard, it is noted that the embodiments disclosed in FIG. 12 presented two-way communication between all of the subsystems. It is noted that in some embodiments, there is only one-way communication between one or more or all of the subsystems. Moreover, in some embodiments, there can be subsystems that do not communicate with each other one way or the other. Any arrangement of medication between the subsystems can be utilized that can enable the teachings detailed herein providing that such is utilitarian value.

In a modification of the embodiment of FIG. 12, the system can include a fourth sub-system. In an exemplary embodiment, the fourth sub-system can be an electrotherapy system. In this regard, in at least some exemplary embodiments, one or more of the prior systems can be utilized to evaluate whether or not a condition associated with the recipient is present, such a condition indicative of an oncoming seizure or the like. Based on this determination, the fourth sub-system can be engaged in an attempt to avoid or otherwise mitigate the effects of the seizure. In an exemplary embodiment, consistent with the other teachings herein, the fourth sub-system can be integrated with the other sub-systems. Alternatively, the fourth sub-system can be a separate device relative to that containing one or more or all of the other sub-systems. In other embodiments, other types of stimulus applying devices can be utilized in addition to her other than an electrotherapy system.

In at least some exemplary embodiments, the second sub-system is part of an environmental classifier and outputs data indicative of a classification of the environment. Again, in some embodiments, environmental classifiers that are known in utilized in the hearing prostheses arts and/or the retinal implants parts can be utilized in whole or in part as part of the second sub-system.

In some embodiments, the second sub-system is included in a prosthesis that is at least partially implantable, while in other embodiments, this is not the case. Still further, consistent with the teachings detailed above, in at least some exemplary embodiments, the first, second and third sub-systems are part of an integrated prosthesis system and/or a part of an integrated medical device, and in some embodiments this is also the case with respect to the fourth-sub system and/or other sub-systems, which can include stimulating systems that are configured to stimulate or otherwise apply some form of energy to the recipient to achieve a certain medical response. Any one or more of the subsystems detailed herein can be or cannot be integrated in a device with respect to any one or more the other subsystems in at least some exemplary embodiments.

In some embodiments, the third sub-system is configured to identify whether the recipient is moving and/or quantify the movement of the recipient based on the output from at least the second sub-system and determine one or more of whether to activate the first sub-system, a level of activation of the second sub-system or a level of activation (which includes whether to activate) a fourth sub-system that applies stimulation to the recipient, based on the identification. Again, as detailed above, in some instances, the teachings detailed herein are directed to purposely not obtaining data or otherwise measurements under certain circumstances. With respect to the issue of the level of activation of the second sub-system, in an exemplary embodiment, the second sub-system is part of a system that is utilized as a sensory prosthesis or otherwise is a sensory prosthesis. In some embodiments, there can be utilitarian value with respect to limiting the amount of stimulation associated with the environment the recipient. For example, if it appears that the recipient might be headed towards a seizure, it might be utilitarian to reduce the amount of sound noise that is being received by and/or applied to the recipient. That said, in some embodiments, the goal is not necessarily to reduce stimulation for treatment purposes, but instead to reduce stimulation for measurement purposes. In this regard, if a sound probe or the like is utilized, where there is utilitarian value with using such in a quiet environment, the system could artificially lower the amount of ambient sound that the recipient is hearing via the utilization of that second sub-system. With respect to the fourth sub-system, this can be a system that is specifically dedicated to the application of stimulation of the recipient for medical purposes, such as electroshock therapy or the like or any other system that can have utilitarian value.

In some embodiments, the third sub-system is configured to identify whether the recipient is in a sensorially noisy environment and/or quantify the sensorial noise in the noisy environment based on the output from at least the second sub-system and determine whether to activate the first sub-system or a level of activation of the second sub-system based on the identification. Again, consistent with the above, the noise can be light and noise or sound noise or smell noise, etc. In some embodiments, phenomenon sensed by the first sub-system is a physiological phenomenon. Further, the third sub-system configured to analyze output from at least the second sub-system and the first sub-system to determine the level of activation of the second sub-system and/or a level of activation of a fourth sub-system, if present, that applies stimulation to the recipient. In some embodiments, the phenomenon sensed by the first sub-system is a physiological phenomenon.

Also, again in some embodiments where the phenomenon sensed by the first sub-system is a physiological phenomenon, the third sub-system is configured to analyze output from at least the second sub-system in isolation from any output, if present, from the first sub-system to determine whether to activate the first sub-system or a level of activation of the second sub-system or a level of activation of a fourth sub-system, if present, that applies stimulation to the recipient based on the identification.

Also, while the embodiments detailed above have been directed towards the possibility of discounting measurements or otherwise not taking measurements, again it is noted that in some other embodiments, the frequency of measurement taking and/or the amount of measurement taking, etc., is actually increased depending on a given scenario. By way of example only and not by way of limitation, it was noted above that measurements can include EKG measurements. In an exemplary embodiment, it could be utilitarian with respect to determining that the recipient is exercising or engaging in activities, or that that might heighten a heart attack occurrence of the like, and thus upon a determination by the given subsystem that the recipient is engaging in such, the number of measurements might be increased.

Note also that while the above is tended to focus on increasing or decreasing or discounting or paying attention to measurements in a semi-binary manner, is also noted that some embodiments can include paying more attention to given measurements. Again, in an example where the system determines that the recipient is beginning to exercise, this could provide an indication to a healthcare professional or the like to more closely monitor the measurements. In an exemplary embodiment, this could indicate that the measurement should be evaluated and/or monitored in real time as opposed to later (later for data collection purposes or otherwise to analyze trends). Indeed, based on the indication of the activity and/or the environments, etc., of the recipient, even automated monitoring and/or analysis systems could be implemented based on a determination associated with the input achieved by the given sub component. Also, holds could be adjusted accordingly. Again, consistent with the concept where the recipient is beginning exercising, abnormalities in the measurements that might otherwise be ignored would be less likely to be ignored upon a determination that the recipient is exercising, because there could be a possibility of increased heart attack, etc. Any data evaluation and/or manipulation process that can be utilized in a utilitarian manner based on the given determinations detailed herein can be utilized in at least some exemplary embodiments.

It is noted that herein, the phrase recipient is sometimes utilized. Any disclosure herein that refers to the recipient corresponds to an equal disclosure of a person whether or not that person is a recipient of a prostheses, unless otherwise noted, providing that the art enables such, and vice versa.

In an exemplary embodiment, one or more of the devices and/or systems and/or subsystems, etc., disclosed herein, and variations thereof, include a processor, which processor of can be a standard microprocessor supported by software or firmware or the like that is programmed to execute one or more of the actions and functionalities herein. The processor can include input and/or output connections. By way of example only and not by way of limitation, in an exemplary embodiment, the microprocessor can have access to lookup tables or the like having data and/or can compare features of the input signal and compare those features to features in the lookup table, and, via related data in the lookup table associated with those features, make a determination about the input signal, and thus make a determination, etc. Numeric analysis algorithms can be programmed in the processors, etc., to implement the teachings herein.

It is noted that the teachings detailed herein can be implemented in any processor-based device that can enable the teachings herein. In an exemplary embodiment, a sensory prosthesis, such as a hearing prosthesis or a light prosthesis, can be modified by adjusting the circuitry or otherwise providing programming to a given processor so as to enable the teachings detailed herein. Further, an Internet of things-based approach can be utilized. Also, various components and systems and subsystems can be network so that some actions and/or functionalities detailed herein are performed by components that are remote and/or geographically distant from other components. Accordingly, the teachings detailed herein can be implemented utilizing the Internet or landline-based devices or wireless communication system such as cellular phone communication systems, etc. Any of the prostheses and/or medical devices detailed herein can correspond to body worn devices or body carried devices. Again, these body worn or body carried devices can have processors that are programmed to receive input and/or to provide output to implement the teachings detailed herein. In some embodiments, programs personal computers and/or laptop computers and/or personal handheld devices, such as smart phones or smart watches etc. can be utilized to execute at least some of the functionalities and method actions detailed herein.

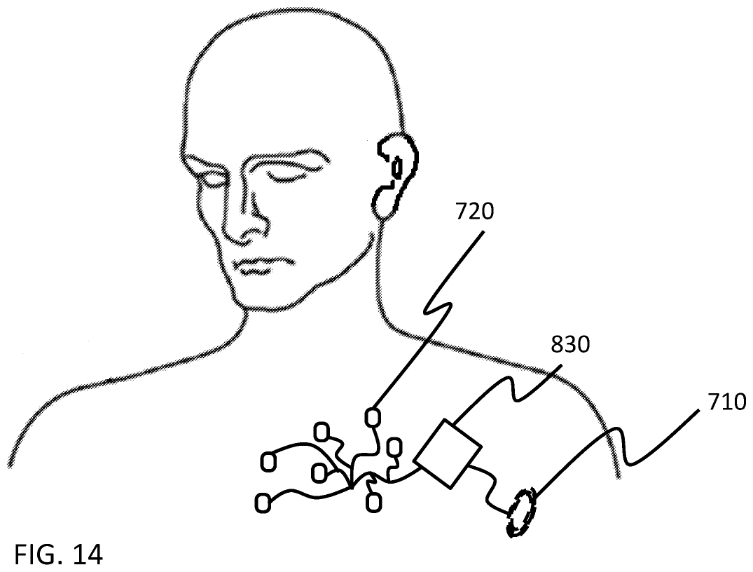

Many of the embodiments detailed above have focused on device that is implanted in the head or otherwise includes an inductance coil that is located in the head. Indeed, the embodiments detailed above have generally focused on a hearing prosthesis, such as a cochlear implant (although it is noted that in at least some other exemplary embodiments, the hearing prosthesis is a DACI prosthesis and/or a middle ear hearing prosthesis and/or an active transcutaneous bone conduction device hearing prosthesis, all of which include an implanted radiofrequency coil such as a coil in the form of an inductance coil or any other coil that can enable the teachings detailed herein, or a radio frequency antenna or any other device that can enable communication—any disclosure herein of a cochlear implant corresponds to a disclosure in an alternate embodiment of one of the other aforementioned hearing prostheses). Some other embodiments can be embodiments that include an implanted component that is implanted elsewhere other than the head. By way of example only and not by way of limitation, in an exemplary embodiment, there can be a heart monitor and/or a heart stimulator (pacemaker), such as by way of example only and not by of limitation, the arrangement seen in FIG. 13. As seen, a heart monitor comprises a plurality of sensor/read electrodes 720, connected to an inductance coil 710 via leads 730. In this embodiment, the implanted device has no recording/storage capabilities, and requires an external device to receive a signal from the implanted inductance coil 710 so as to retrieve in real time the signal therefrom. Not shown is an implantable component that converts the electricity sensed by the sensor/read electrodes into a signal that is transmitted by the inductance coil 710. In an exemplary embodiment, the sensor arrangement seen in FIG. 7 is an implanted EKG sensor arrangement. FIG. 14 depicts another arrangement of an implantable sensor arrangement that again includes the sensor/read electrodes 720 and the leads 730. Here, in this embodiment, there is a housing 830 which includes circuitry that is configured to receive the signals from the leads from the electrodes 720 and record the data therefrom or otherwise store the data, and permits the data to be periodically read from an external device when the external device comes into signal communication with the implanted inductance coil 710. Alternatively, and/or in addition to this, the circuitry is configured to periodically energize the inductance coil 710 so as to provide the data to the coil 710 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EKG sensor arrangements can be obtained and modified so as to implement the teachings detailed herein and/or variations thereof.

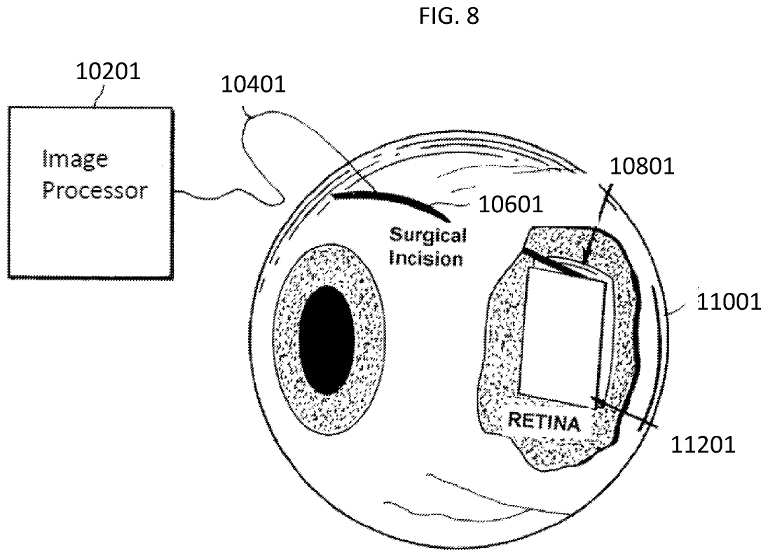

It is noted that some embodiments of the sensor arrangement of FIG. 8 includes an implanted battery or otherwise implanted power storage arrangement, while in other embodiments the arrangement specifically does not, making the arrangement akin to the embodiment of FIG. 13.

In view of the above, it can be seen that the aforementioned measurements can also correspond to EKG measurements or the like. In this regard, there can be utilitarian value with respect to determining whether or not the recipient is exercising or the like so as to discount or otherwise not even monitor (or alternatively, monitor more carefully more frequently, etc., the EKG measurements).

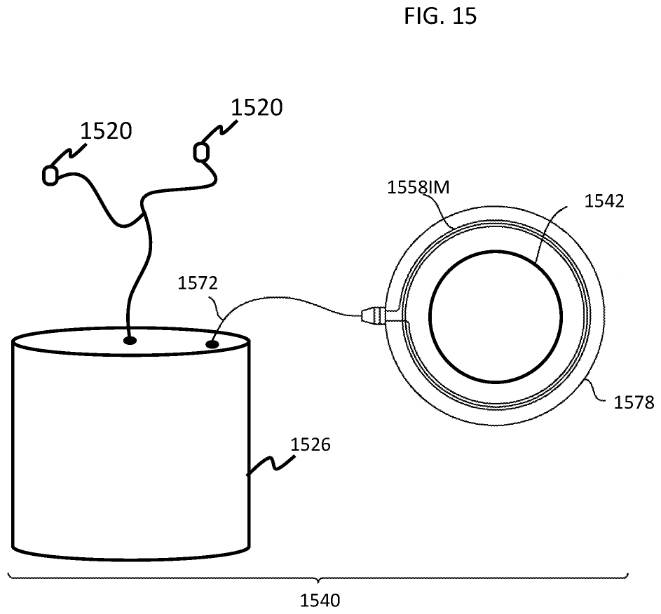

FIG. 15 presents another exemplary embodiment of an implantable device, which implantable device can be utilized to obtain measurements that can be applicable in some embodiments of the teachings detailed herein. With respect to the implantable device, FIG. 15 provides an exemplary functional arrangement of an implantable device 1540 that is configured to transcutaneously communicate via an inductance field with the external device of FIG. 14 or an analogous device. Implantable component 1540 can correspond to the implantable component of the system 10 of FIG. 1. Alternatively, and/or in addition to this, the implantable component of FIG. 15 can correspond by way of representation to the implantable component of the EEG embodiment or the EKG embodiment or the retinal implant embodiment. As can be seen, external component 1540 includes an implantable housing 1526 which is connected via cable 1572 to an exemplary implanted coil apparatus 1578 including an implanted inductance coil 1558IM, corresponding to the external coil of FIG. 1 in this exemplary embodiment, where FIG. 15 represents the cochlear implant of FIG. 1. As illustrated, the implantable component 1540 comprises an implanted inductance communication assembly that includes the coil 1558IM and a magnet 1542. This magnet 1152 interacts with the external magnet of the implantable component to hold the headpiece 1478 against the skin of the recipient. In an exemplary embodiment, the implantable component 1540 is configured to transmit and/or receive magnetic data and/or receive power transcutaneously via coil 1558IM from the external component, which includes an inductance coil as detailed above. The coil 1558IM is electrically coupled to the housing 1526 via cable 1572. The housing 1526 may include may include, for example, at least some of the components of the implantable component of FIG. 1, such as for example, the stimulator of the cochlear implant where the embodiment of FIG. 15 represents such.

Implantable component 1540 also includes a stimulating assembly which includes leads extending from the housing 1526 that ultimately extend to electrodes 1520, as seen. In the embodiment where FIG. 15 represents the implantable component of the cochlear implant, electrodes 1520 and the associated leads functionally represents the electrode assembly of a cochlear implant, although it is specifically noted that in a real cochlear implant, electrodes 1520 would be supported by a carrier member instead of being “free” as shown. That said, in an exemplary embodiment, FIG. 15 can represent the EEG and/or the EKG systems detailed above, where the electrodes 1520 are read/sense electrodes. Still further, in an exemplary embodiment, the implantable component of FIG. 15 can represent the retinal implant. Note further, that in an exemplary embodiment, the electrodes 1520 are replaced with mechanical actuators, and thus the embodiment of FIG. 15 represents an active transcutaneous bone conduction device and/or a middle ear implant, etc.

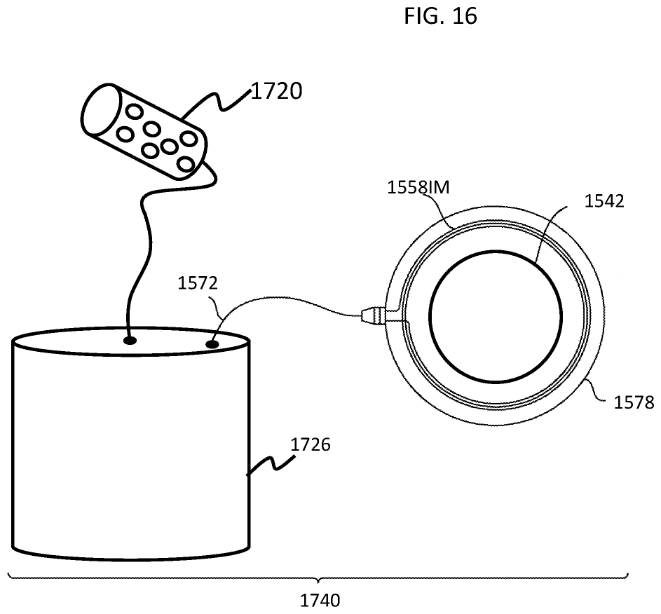

In this regard, FIG. 15 is presented for conceptual purposes to represent how the external component of FIG. 4 communicates with the implanted component. Along these lines, in an exemplary embodiment, the external component's magnet magnetically aligns with the implantable component's magnet, thus aligning the external coil with the implanted coil. This can have utilitarian value as aligning the coils provide efficiency relative to that which would be the case if the coils are misaligned. By way of example only and not by way of limitation, in an exemplary embodiment, the magnets are disk magnets having the north-south polarity aligned with the axis of rotation of the disks. In this regard, the magnets want to align the magnetic fields with one another, and thus by holding the respective coils at predetermined and control distances from the respective magnets utilizing the structure of the external component and/or the implantable components (e.g., a silicone body) the coils will become aligned with each other because the magnets will become aligned with each other. FIG. 16 depicts how the respective magnets aligned with one another with respect to their north south poles. As can be seen, both magnets aligned about axis 1690. This has the effect of aligning the respective coils.

Accordingly, in an exemplary embodiment, implantable component 1540 can be utilized with the external component that is an external component of a hearing prosthesis and/or an external component of a retinal implant and/or an external component of a sense prostheses as detailed herein.

An exemplary embodiment includes an implantable EEG monitor or another type of monitor with an internal power supply that operates in two distinct operation modes. One of the modes is for day use where the recipient is conscious and/or active. The day mode can be such that the implanted component operates autonomously without any external component, although in some embodiments, the implantable component can also operate in the day mode with an external component. Concomitant with the teachings detailed above, the day mode can be such that the implanted component receives power only from an implanted battery or other power source that is implanted in the recipient. In this exemplary embodiment, the implant monitors and/or stores data, such as EEG and/or EKG data, during the day mode of operation. Also, in at least some exemplary embodiments, during the day mode of operation, the implantable component can analyze the data, and can make a determination as to whether or not an alarm should be provided to the recipient based on the data. In an exemplary embodiment, the alarm is provided according to the teachings detailed herein utilizing componentry all of which is implanted in the recipient.

It is noted that in some embodiments, where there is a day or night mode, the teachings detailed herein can be utilized to transition the device from one mode into another, based on the data obtained by the second subsystem or the like.

Teachings detailed herein can be applicable to management or otherwise the monitoring of epilepsy prone peoples. In this regard, seizure events can be infrequent, with many months between events. Diagnosis requires at least one seizure to be captured. Many patients remain undiagnosed or incorrectly diagnosed due to lack of long term monitoring. Utilizing the teachings detailed herein, as can be seen, can provide EEG data capturing prior to and/or during a seizure. Accordingly, some exemplary methods include practicing the details herein respect to a method of treating and/or monitoring epilepsy.

It is noted that while the embodiments detailed herein have focused on electrical detection/electrical monitoring/electrical analyses (ECE/EEG), other embodiments are related to detecting/monitoring, analyzing changes in the chemical composition of substances inside a body. By way of example only and not by way of limitation, FIG. 16 provides a schematic of an implantable component 1740 that is configured to monitor body fluid chemistry. In this regard, there is housing 1726 that includes a processor or the like that is program to analyze data via a signal from blood capture device 1720. The blood capture device 1720 is configured to capture blood and/or to analyze the blood to evaluate the chemistry thereof. By way of example only and not by way of limitation, the implantable component 1740 can be a blood glucose implant monitor that monitors blood directly or indirectly to determine its glucose level. The captured blood then is analyzed by a device 1726.

Note further that in an exemplary embodiment, the implantable component 1740 can be a new drug analyzer. By way of example only and not by way of limitation, the implantable component 1740 can be configured or otherwise programmed to analyze blood chemistry to evaluate the effects of a new drug.

The above said, it is noted that in at least some exemplary embodiments, an EEG system can be utilized to evaluate blood glucose levels and/or new drug efficacy. In this regard, there can be a scenario of use where there is a new drug introduction, and the evaluation regime of the new drug introduction includes brain monitoring, where the brain monitoring includes application of an EEG monitoring. At least some of the exemplary embodiments detailed herein provide enablement for continuous monitoring, and such can be very utilitarian for new drug evaluation.

It is briefly noted that a tertiary monitoring method through EEG analysis can detect hypoglycemia (low blood sugar levels). To maximize utilitarian value, the implantable component can be monitored continuously, and long term.

Traditionally, the problem associated with monitoring the above-noted phenomenon is that if the data is to be streamed in real-time or semi-real-time, an external component is required. Again, typically, the external component is an external component that is worn on the head. During sleep or a seizure though, this component would often be removed, or fall off. Accordingly, the teachings detailed herein can provide for the streaming and/or the recordation of the data in the complete absence of the traditional external component that is utilized with the implant.

It is specifically noted that in at least some exemplary embodiments, the implantable apparatus is not a hearing prosthesis as that would be understood by the person of ordinary skill in the art. In this regard, simply because the device evokes a hearing percept does not mean that it is a hearing prosthesis. As used herein, the phrase hearing prosthesis means that the device is configured to capture sound and evoke a hearing percept based on the captured sound. The teachings detailed herein that utilize a hearing percept to provide an indication to the recipient specifically do not require captured sound. In this regard, the implantable component is preprogrammed and/or preconfigured to evoke only a limited number of hearing percepts irrespective of the environment.

That said, in at least some exemplary embodiments, the teachings detailed herein can be combined with a hearing prosthesis or otherwise are even limited to a hearing prosthesis. In this regard, in an exemplary embodiment, the implantable component is an implantable component of a hearing prosthesis that includes a tissue stimulator that provides the indication.

In an exemplary embodiment, the implantable component includes a tissue stimulator that provides the indication. The tissue stimulator can be part of an apparats that provides additional functionality beyond (i) stimulating tissue to provide the indication (e.g., the system can be an EEG monitor, an EKG monitor, a body fluid monitor, a drug efficacy monitor, etc.) and (ii) if the implantable component is configured to provide functionality of a hearing prosthesis, stimulating tissue to provide a hearing percept based on external stimulation. External stimulation includes sound captured by sound capture apparatus, streamed audio to the hearing prosthesis, etc.

In an exemplary embodiment, the implantable component is part of a body monitoring device configured to monitor aspects of a recipient's body, wherein the implantable component is configured to evaluate the monitored aspects and determine if an aspect is outside of a given parameter, and upon such determination, provide the indication to the recipient, wherein the indication is an indication that an aspect is outside of a given parameter. Again, as detailed above, in an exemplary embodiment, the EEG monitor and monitor signals for a potential seizure or the like. The implantable component can analyze the signals in real time or near real time, and if the signals are indicative of a potential seizure, alert the recipient by providing the indication, which indication would be a warning that a seizure could be imminent.

FIG. 8 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. An apparatus, comprising:
a medical device, wherein the medical device is configured to determine, based on non-movement data associated with a recipient of the medical device, whether or not a data collection activity should be commenced, wherein the data is physiological data associated with the recipient of the medical device.

2. The apparatus of claim 1, wherein:
the medical device is a hearing prosthesis; and
the hearing prosthesis is configured to evaluate a sound environment of the hearing prosthesis to determine whether or not the data collection activity should be commenced.

3-4. (canceled)

5. The apparatus of claim 1, wherein:
the medical device includes an implantable component configured to execute acoustic probing of the recipient's body.

6-7. (anceled)

8. The apparatus of claim 1, wherein:
the medical device is also configured to sense a phenomenon indicative of movement of the recipient; and
the medical device is configured to evaluate the sensed phenomenon indicative of movement to determine whether or not the data collection activity should be commenced.

9-11. (canceled)

12. The apparatus of claim 1, wherein:
the medical device includes a first sensor system and a second sensor system;
the first sensor system is the sensor system used to collect the data upon data collection commencement;
the second sensor system collects non-physiological data; and
the medical device is configured to evaluate the collected non-physiological data to make the determination.

13. (canceled)

14. The apparatus of claim 1, wherein:
the medical device is configured to determine and/or extrapolate a state of a recipient of the medical device; and
the medical device is configured to determine whether or not the data collection activity should be commenced based on the determination of the state of the recipient.

15-19. (canceled)

20. The apparatus of claim 1, wherein:
the medical device is configured to determine whether or not to initiate active probing of the recipient.

21-26. (canceled)

27. A system, comprising:
a first sub-system configured to sense a phenomenon associated with an individual;
a second sub-system configured to at least one of capture sound, capture light, or capture electromagnetic radiation; and
a third sub-system configured to at least one of:
analyze output from at least the second sub-system and determine at least one of whether to activate the first sub-system or a level of activation of the second sub-system; or
analyze output from at least the second sub-system and the first sub-system and determine at least one of whether to activate or a level of activation of a fourth sub-system that stimulates the recipient.

28. The system of claim 27, wherein:
the first sub-system is an EEG monitor.

29. The system of claim 27, wherein:
the system is configured to analyze at least the output from at least the second sub-system and identify at least one of a locational situation of the recipient or an activity in which the recipient is engaged or a state of the recipient; and
the system is configured to make the determination(s) based on the identification.

30. (canceled)

31. The system of claim 27, wherein:
the second sub-system is part of an environmental classifier and outputs data indicative of a classification of the environment.

32-33. (canceled)

34. The system of claim 27, wherein:
the third sub-system is further configured to identify whether the recipient is moving and/or quantify the movement of the recipient based on the output from at least the second sub-system and determine one or more of whether to activate the first sub-system, a level of activation of the second sub-system or a level of activation of a fourth sub-system that applies stimulation to the recipient based on the identification.

35. (canceled)

36. The system of claim 27, wherein:
the phenomenon is a physiological phenomenon; and
the third sub-system is configured to analyze output from at least the second sub-system and the first sub-system to determine the level of activation of the second sub-system and/or a level of activation of a fourth sub-system, if present, that applies stimulation to the recipient.

37. The system of claim 27, wherein:
the phenomenon is a physiological phenomenon; and
a third sub-system configured to analyze output the second sub-system in isolation from any output, if present, from the first sub-system to determine whether to activate the first sub-system or a level of activation of the second sub-system and/or a level of activation of a fourth sub-system, if present, that applies stimulation to the recipient.

38. A method, comprising:
obtaining first data indicative of an occurrence associated with a recipient of a prosthesis utilizing a device of the recipient, wherein the prosthesis is effectively stationary with respect to a local position at the time that the first data is obtained; and
determining whether to at least one of implement measuring involving the recipient or to discount second data involving the recipient based on the obtained first data.

39. The method of claim 38, wherein:
the measuring is a high-fidelity recording; and
low fidelity recording has already taken place upon the action of determining.

40. The method of claim 38, wherein:
the measuring is a high-fidelity recording;
low fidelity recording is occurring at the time of the determination; and
the action of determining includes determining to implement the high fidelity measuring, and thus transition from low fidelity measuring to high fidelity measuring.

41-44. (canceled)

45. The method of claim 38, wherein:
the first data is indicative of at least one of an environment of the recipient, an activity engaged in by the recipient or a state of the recipient;
the method includes evaluating the first data and determining based on the evaluation that the environment, the activity and/or the state is indicative of deleteriousness to a utilitarian value of the measuring; and
the action of determining includes discounting the second data based on the evaluation.

46. The method of claim 38, wherein:
the first data is obtained at plurality of times over a first temporal period;
the determining is respectively executed a plurality of times for the respective obtained first data;
the obtained first data includes a respective data indicative of a respective sensorially noisy environment and a respective determining of the determining action includes respectively determining to not implement measuring temporally correlated to the respective obtained first data and/or to discount the respective second data temporally correlated to the respective obtained first data;
the obtained first data includes a respective data indicative that the noisy environment is no longer present and respective determining includes determining to implement the measuring temporally correlated to the respective obtained first data or to not discount the second data temporally correlated to the respective obtained first data.

47. (canceled)

48. The method of claim 38, wherein:
the obtained first data includes first sub-data obtained during a first temporal period when the recipient is experiencing a first classification of occurrence associated with the recipient;
the obtained first data includes second sub-data obtained during a second temporal period after the first temporal period when the recipient is experiencing a second classification of occurrence associated with the recipient; and
the determining includes:
a first determining to implement measuring temporally correlated to the first sub-data or to not discount third sub-data included in the second data; and
a second determining to halt measuring temporally correlated to the second sub-data or to discount fourth sub-data included in the second data.

49-56. (canceled)
